(19) United States
(12) Patent Application Publication
(10) Pub. No.: US 2025/0329454 A1
(43) Pub. Date: Oct. 23, 2025

(54) DIAGNOSTIC INDICES FOR NEURODEGENERATIVE CONDITIONS

(71) Applicant: Chase Therapeutics Corporation, Washington, DC (US)

(72) Inventors: Thomas N. CHASE, Washington, DC (US); Kathleen CLARENCE-SMITH, Washington, DC (US)

(21) Appl. No.: 18/290,569

(22) PCT Filed: Jun. 15, 2022
(86) PCT No.: PCT/US2022/033517

§ 371 Date: Nov. 14, 2023

Related U.S. Application Data
(60) Provisional application No. 63/210,939, filed on Jun. 15, 2021.

(51) Int. Cl. G16H 50/20; C12Q 1/48; C12Q 1/6883; G01N 33/68; G16H 20/10; G16H 50/30; G16H 50/70

(52) U.S. Cl. CPC G16H 50/20; C12Q 1/485; C12Q 1/6883; G01N 33/6896; G16H 20/10; G16H 50/30; G16H 50/70; C12Q 2600/106; C12Q 2600/112; C12Q 2600/178; G01N 2333/4709; G01N 2333/912; G01N 2800/2835; G01N 2800/52

(57) ABSTRACT

A method to evaluate individuals with certain neurodegenerative diseases (e.g., Parkinson's Disease) in relation to etiologic diagnosis, prognosis and response to therapy involving the noninvasive collection of a biologic sample (e.g., venous blood), isolation of small, neuronally-derived, extracellular vesicles (e.g., exosomes), assay of their external and/or internal contents for quantities of informative biomarkers (e.g., signaling kinases, catalytic proteins and miRNA species) for the construction of a diagnostic/prognostic/response algorithms of clinical utility.

### REFERENCE TO RELATED APPLICATIONS

This application is a 371 U.S. National Stage Entry of PCT/US2022/033517, filed Jun. 15, 2022, and claims priority to U.S. provisional application 63/210,939, filed Jun. 15, 2021, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 10, 2024, is named CHASE-006-US-NTL_SL.txt and is 837 bytes in size.

### BACKGROUND

Neurodegenerative diseases are characterized by degenerative changes in the brain, including loss of function and death of neurons. Neurodegenerative diseases include, without limitation, Parkinson's disease, Alzheimer's disease, Huntington's disease, amyotrophic lateral sclerosis and Lewy Body dementia.

Various signaling kinases have been implicated in neurodegenerative diseases. See, for example, Mehdi, S. J. et al., “Protein Kinases and Parkinson's Disease,” Int J Mol Sci. 2016 September; 17 (9): 1585 (doi: 10.3390/ijms17091585); Martin, L. et al., “Tau protein kinases: Involvement in Alzheimer's disease,” Ageing Research Reviews, Volume 12, Issue 1, January 2013, Pages 289-309 (doi.org/10.1016/j.arr.2012.06.003); and Bowles, K. R. et al., “Kinase Signaling in Huntington's Disease,” Journal of Huntington's Disease 3 (2014) 9-123 (DOI 10.3233/JHD-140106).

Many neurodegenerative diseases are characterized by the aberrant accumulation of oligomeric forms of proteins. It is believed that these oligomeric forms contribute to neuronal degeneration and death. In particular, Parkinson's Disease is characterized by accumulation of oligomeric forms of alpha synuclein. It has further been found that alpha synuclein can aggregate to form co-polymers with other proteins, such as tau and amyloid beta.

### SUMMARY OF THE DISCLOSURE

Referring to FIG. 1, assays for kinases include the following operations: A body fluid sample, such as a blood or saliva sample from a subject is obtained (100). The blood sample may be treated to provide a blood fraction, e.g., a plasma sample (110). The blood sample is enriched for extracellular vesicles, e.g., exosomes. This can be a two-step operation that involves, first, isolating total exosomes (111) and, second, enriching for neuronally derived exosomes (112). Neuronally derived exosomes can be those from all neurons, generally (120a), or specifically those from a subset of neurons, such those using dopamine as their neurotransmitter (112b).

Isolated exosomes can be processed in three ways. In one method a total exosomal lysate is used. In another method, the internal exosomal contents or cores are isolated and enriched, for example by permeabilization and washing before use. This can involve scrubbing to remove proteins attached to their surfaces (121). In another method, the membrane contents of the extracellular vesicle are isolated.

The exosomal products are then subject to further analysis (122). Analysis involves measuring in the sample biomarkers selected from either: (i) a plurality of different signaling kinases; and (ii) biomarkers from at least two groups selected from: (1) one or more enzymes selected from signaling kinases and/or catalytic enzymes, (2) one or more neurodegeneration-associated proteins in monomeric or oligomeric form, and (3) one or more miRNAs. Measures of these biomarkers can be used in diagnostic testing to determine presence or absence of, or risk of developing, a particular neurodegenerative condition (e.g., a synucleinopathic condition) or of its cumulative severity or current rate of progression, or to determine efficacy of a drug to alter amounts or relative amounts of one or more biomarker proteins described herein toward normal amounts. Assays can be performed using Western blot or Eliza methodology.

Disclosed herein are, among other things, biomarker profiles for neurodegenerative conditions, such as synucleinopathic conditions, amyloidopathic conditions, tauopathies and Huntington's disease, and the neurodegeneration associated therewith. In certain embodiments, the biomarker profiles comprise measures of a set of biomarkers that include at least one signaling kinase and that can be selected from (1) at least one signaling kinase and, optionally, at least one oligomeric form of a neurodegeneration-associated protein, or (2) each of one or a plurality of different signaling kinases. Biomarker profiles can comprise measures of one or more oligomeric forms of neurodegeneration-associated proteins, such as alpha-synuclein, amyloid beta, tau or huntingtin.

Signaling kinases measured can be one or a plurality of kinases. They can be selected from the same signaling pathway, such as the AKT or mTOR pathway, or from different signaling pathways.

Oligomeric forms of neurodegeneration-associated proteins measured can be a collection of forms, such as total oligomeric alpha synuclein, or individual oligomeric forms, such as a hexamer of alpha synuclein. Alternatively, a plurality of forms can be measured, such as alpha synuclein oligomers in the range of pentamers to partially soluble filaments-mers. Monomeric forms of the neurodegeneration-associated protein also can be measured. So, for example, the biomarker profile can comprise measures of each of one or a plurality of neurodegeneration-associated protein forms selected from: (I) at least one oligomeric form; (II) a plurality (e.g., pattern) of oligomeric forms; (III) at least one oligomeric form and at least one monomeric form; (IV) a plurality of oligomeric forms and at least one monomeric form; (V) at least one oligomeric form and a plurality of monomeric forms; and (VI) a plurality of oligomeric forms and a plurality of monomeric forms.

Further disclosed herein are methods of developing pharmaceuticals for treatment of neurodegenerative conditions, such as synucleinopathic conditions, amyloidopathic conditions, tauopathic conditions, and Huntington's disease. The methods involve using a biomarker profile to determine the effect of a candidate pharmaceutical on the condition. The biomarker profile includes measures of a biomarker set including biomarkers selected from (1) at least one signaling kinase and, optionally, at least one oligomeric form of a neurodegeneration-associated protein, or (2) each of one or a plurality of different signaling kinases. Biomarker proteins can be quantified from, e.g., neuronally-derived extracellular vesicles, e.g., exosomes from the blood of a subject.

In certain embodiments, the protein species are measured from neuronally derived extracellular vesicles (e.g., exosomes) isolated, e.g., from blood, saliva, or urine. The species examined can derive from an internal compartment of the exosome extracellular vesicle, e.g., from exosomes extracellular vesicles from which surface proteins have been removed. The biomarker profiles, measured in this way, represent a relatively simple and non-invasive means for measurement of exosomes contents deriving mainly from the central nervous system.

As such, methods of this disclosure for measuring a biomarker profile for a neurodegenerative condition are useful in drug development for testing neuroprotective efficacy of a drug candidate, sometimes referred to herein as a putative neuroprotective agent. For example, the methods described herein can be used to further understand the downstream effects of kinase activity, and to accelerate the development of effective therapeutic strategies by rapidly and reliably providing quantitative treatment-response information much sooner than by means of currently available methods of clinical evaluation. Bioassay methods also are useful for identifying subjects for enrollment in clinical trials and for determining a diagnosis, prognosis, progression or risk of developing a synucleinopathic condition. Further provided herein are novel methods of treating a subject determined, by the methods of this disclosure, to have or to be at risk of developing neurodegeneration associated with synucleinopathic conditions, in particular, a neuroprotective treatment.

Other objects of the disclosure may be apparent to one skilled in the art upon reading the following specification and claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a flow diagram of an exemplary method detecting kinases and, optionally, neurodegeneration-associated protein forms from extracellular vesicles.

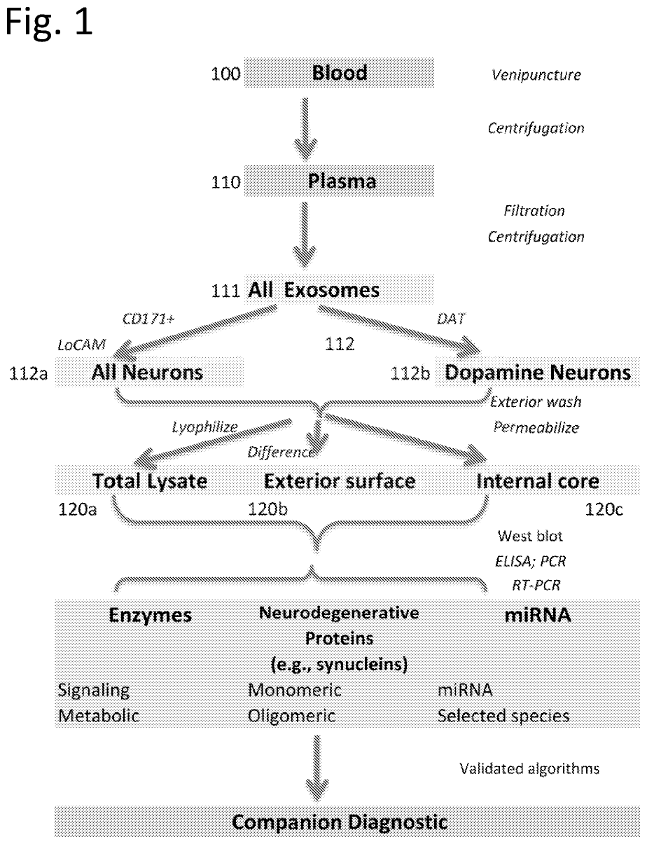

FIG. 2 shows a flow diagram of an exemplary protocol to validate efficacy of a therapeutic intervention.

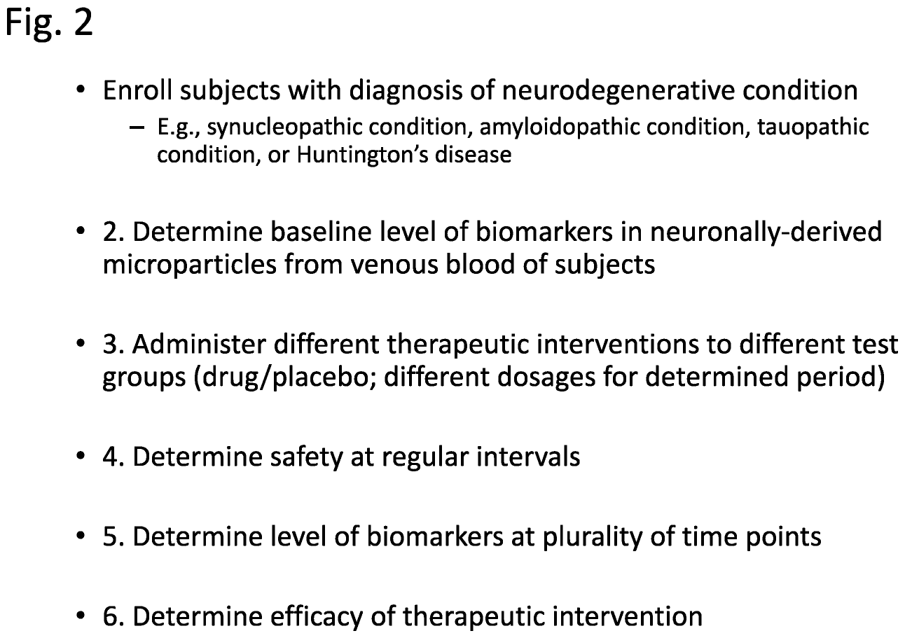

FIG. 3 shows an exemplary flow diagram of creating and validating a diagnostic model for diagnosing a neurodegenerative condition.

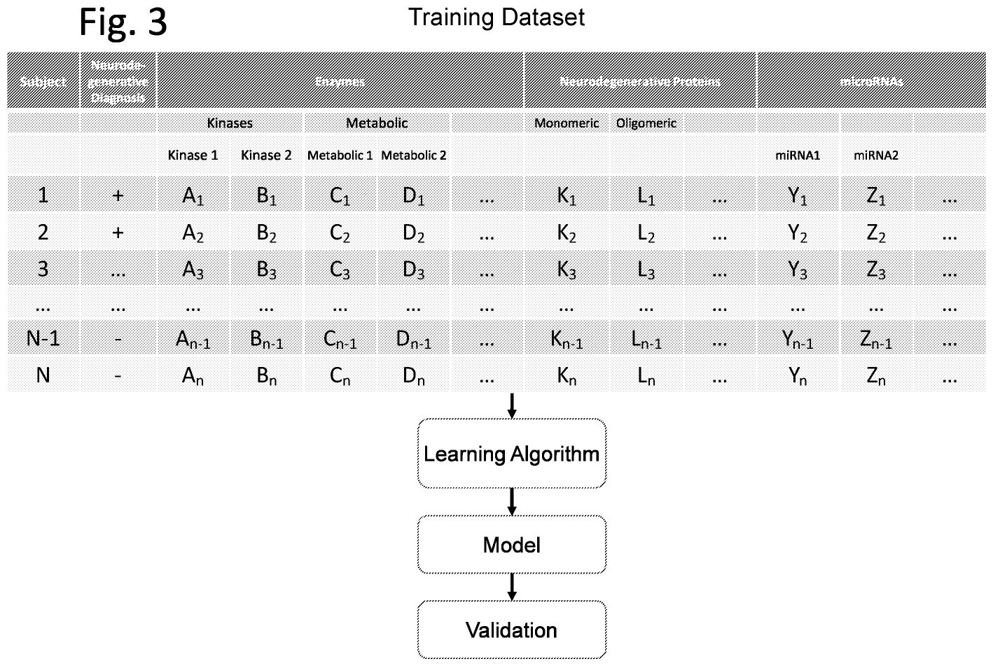

FIG. 4 shows an exemplary flow diagram for classifying a subject according to any of several states by executing a diagnostic algorithm, or model, on a biomarker profile.

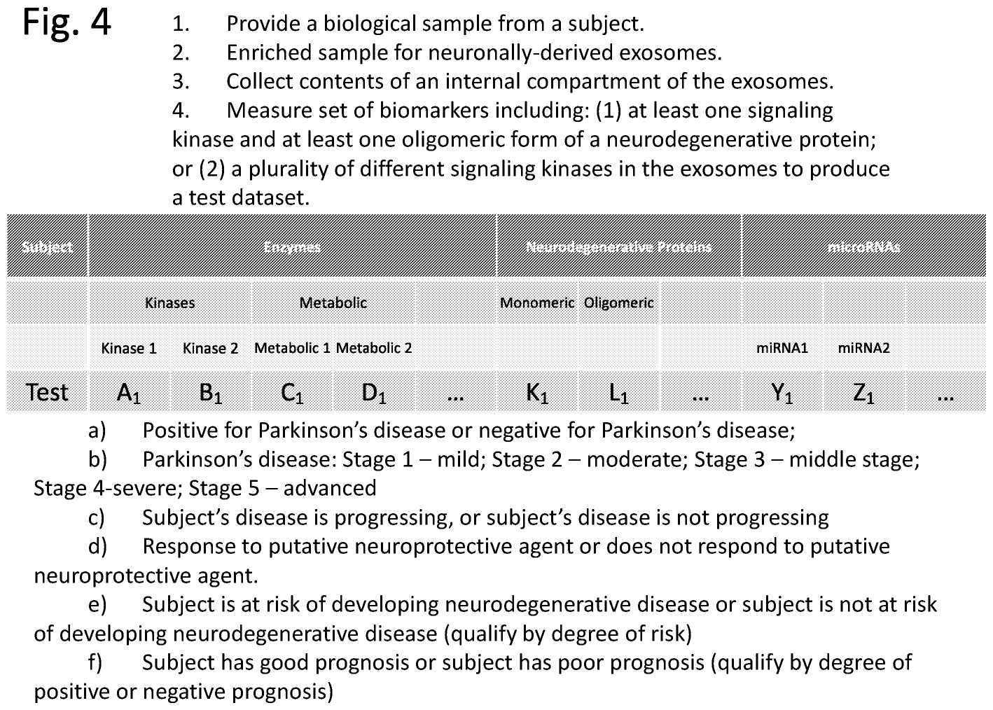

FIG. 5 shows a signal transduction mechanisms implicated in the pathogenesis of Parkinson's Disease.

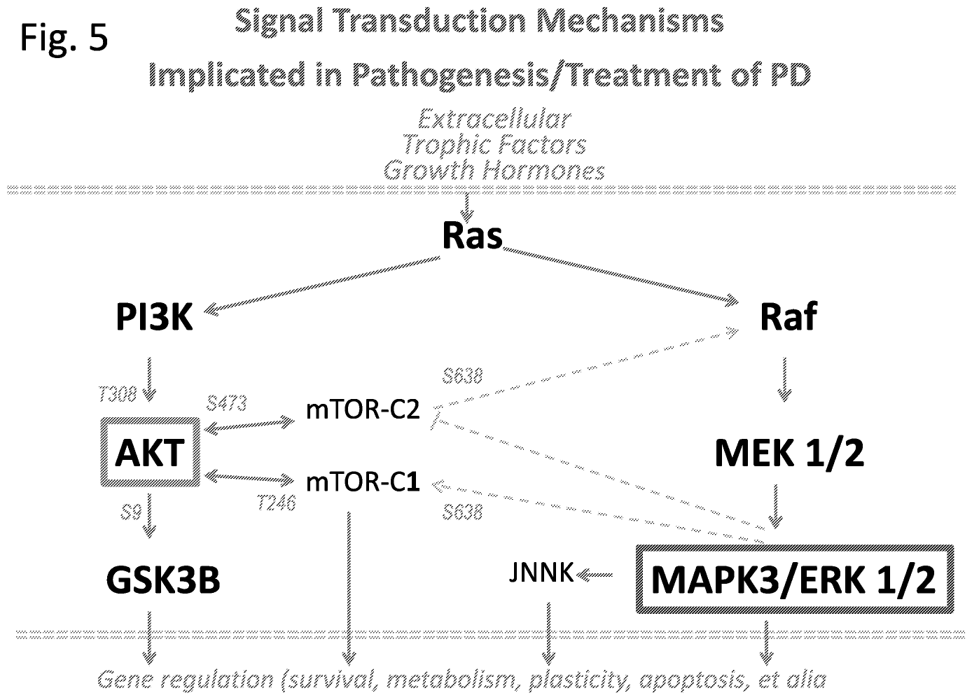

FIG. 6 shows graphs showing that AKT S473 is up-regulated in Parkinson's Disease and MAPK T202 is down-regulated in Parkinson's Disease.

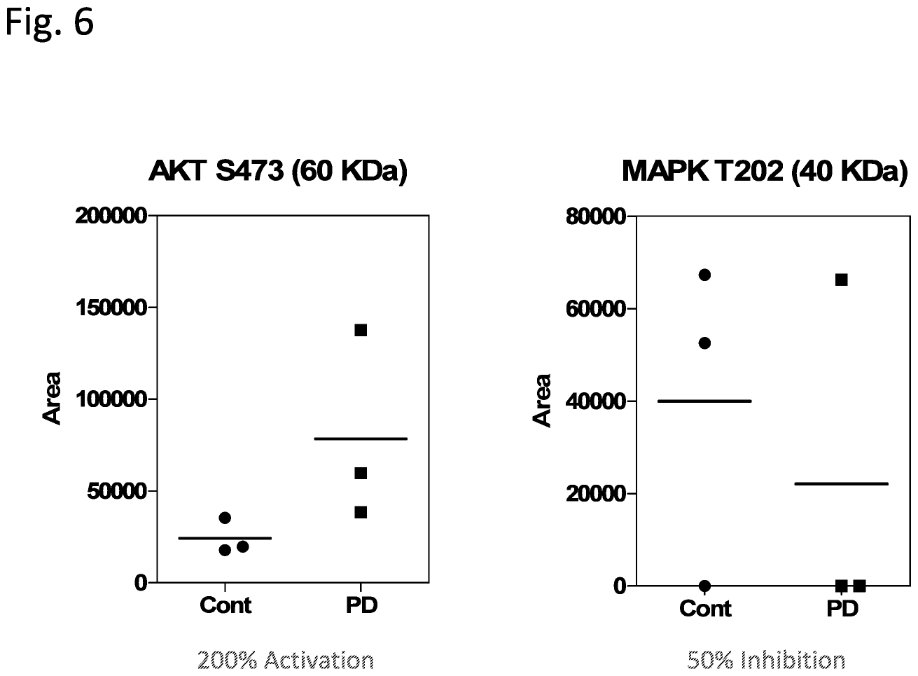

FIG. 7 shows an exemplary indices for neurodegenerative conditions.

FIG. 8 shows an exemplary computer system.

### DETAILED DESCRIPTION OF THE DISCLOSURE

### I. Biomarkers for Neurodegenerative Conditions

Methods disclosed herein are useful for diagnosis of and drug development for a variety of neurodegenerative conditions. These include, without limitation, synucleinopathies (e.g., Parkinson's disease, Lewy body dementia, multiple system atrophy), amyloidopathies (e.g., Alzheimer's disease), tauopathies (e.g., Alzheimer's disease, Progressive supranuclear palsy, Corticobasal degeneration), and Huntington's disease.

### A. Biomarkers and Biomarker Profiles

Biomarkers are analytes that are associated, positively or negatively, alone or in combination, with a particular condition. Analytes that can function as biomarkers include any biological molecule or organic or inorganic molecule that is detectable in a subject or a subject sample. Biological molecules that can serve as biomarkers include, without limitation, polypeptides and polynucleotides, including, for example, proteins and peptides, and nucleic acids, such as RNA and DNA.

As used herein, the term “biomarker” refers to a feature whose measure is associated with a particular biological category. For example, the biomarker may up-regulated or down-regulated in a certain neurodegenerative disorder. The features are typically biomolecules, such as proteins or nucleic acids (e.g., alpha-synuclein, beta-amyloid, protein kinases, miRNA) but they also can be non-molecular features such as a clinical variables (e.g., presence or absence of tremors or of dementia) or phenotypical traits. As used herein, the term “biomarker profile” refers to measures of each of one or a plurality of biomarkers. Biomarker profiles include a plurality of biomarkers may be more closely associated with a particular biological category (e.g., neurodegenerative condition) than single biomarkers alone. Biomarker profiles can include measures of activity of one or a plurality of different signaling kinases, catalytic enzymes, neurodegeneration-associated proteins and/or miRNAs.

Depending on the context in which it is used, the term “biomarker profile” may also refer to a particular pattern of measures of biomarkers that are associated with the category, such as a diagnosis, stage, progression, rate, prognosis, drug responsiveness and risk of developing a neurodegenerative condition. Such measures can be combined into a single index for the condition.

A measurement of a variable, such as kinase activity, can be any combination of numbers and/or words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, such a “healthy” or “unhealthy”, “old” or “young”, “form 1” or “form 2”, “subject 1 . . . subject n,” etc. Measurements of a variable on an ordinal scale produce a ranking, such as “first”, “second”, “third”; or “youngest” to “oldest”, or order from most to least. Measurements on a ratio scale include, for example, any measure on a pre-defined scale, such as mass, signal strength, concentration, age, etc., as well as statistical measurements such as frequency, mean, median, standard deviation, or quantile. Measurements on a ratio scale can be relative amounts or normalized measures. For example, in one embodiment, a biomarker profile comprises a relative amount of a first and second signaling kinase. In another embodiment a biomarker profile comprises a ratio of amounts of two different biomarker proteins.

Abnormal profiles (e.g., abnormal absolute or relative amounts of various signaling kinases) indicate pathologic activity (or a characteristic bodily response to a pathogenic process), and thus time to future clinical onset and subsequent rates of clinical progression. Moreover, return toward normal in biomarker profiles (e.g., reductions in absolute or relative amounts of signaling kinases and/or oligomeric forms of neurodegeneration-associated proteins) reflects the efficacy of a candidate neuroprotective intervention. Accordingly, the biomarker profiles described herein are useful for determining efficacy of drug candidates for their neuroprotective effect. As a practical matter, they may be considered essential to the practical conduct of neuroprotective drug trial in view of savings in both time and cost as well as a definitive means to quantified efficacy against a pathogenic process rather than its clinical manifestations.

Accordingly, biomarker profiles function not only as a diagnostic of an existing pathological state but also as a sentinel of pathology before clinical onset, e.g., when a subject is pre-symptomatic or preclinical, e.g., has signs or symptoms that are insufficient for a diagnosis of disease. This is relevant since the relative success of neuroprotective treatments often appear related to their earliest possible administration. Further, it is believed that these biomarker profiles indicate the stage (e.g., rate of or cumulative amount of neuronal loss) of a neurodegenerative condition. Accordingly, determining biomarker profiles can be of critical importance for determining effectiveness of a treatment, for example, in clinical trials and, for therapeutic interventions believed to be effective for treating neurodegeneration including, e.g., synucleinopathy, amyloidopathy, tauopathy or Huntington's disease in the individual.

Furthermore, bioassay-derived indices/indexes contribute to advancing understanding of the pathogenesis of neurodegenerative disease. A more precise understanding of disease mechanisms, possibly differing between patients with similar clinical phenotypes, will help guide future efforts towards developing more specific and thus more effective therapeutic interventions.

### B. Enzymes

Neurodegenerative conditions are characterized by abnormal changes in the activity (increased or decreased) of particular enzymes, including signaling kinases and catalytic enzymes. Measuring activity of these signaling kinases in a subject can be used for diagnosis, prognosis, patient progress, patient stratification and drug development and testing.

### Signaling Kinases

Kinases include any kinase involved in signaling pathway.

Kinases associated with Parkinson's disease or the administration of medications that influence of the symptoms of Parkinson's disease (e.g., pramipexole (6-propylamino-4,5,6,7-tetrahydro-1,3-benzothiazole-2-amine)) include, without limitation, mTOR (mechanistic target of rapamycin), mitogen-activated protein kinase (MAPK or MEK), extracellular signal-regulated kinases (ERK), glycogen synthase kinase 3 beta (GSK3B), AKT kinase and beclin Leucine-Rich Repeat Kinase 2 (LRRK2), members of the c-Jun N-Terminal Kinase Signaling Pathway (JNK) (MAPK serine-threonine kinases), and Phosphatase and Tensin Homolog (PTEN)-Induced Putative Kinase 1 (PINK1).

Kinases associated with Alzheimer's disease include, without limitation, Tau protein kinases such as proline-directed protein kinases (PDPK), protein kinases non-PDPK and tyrosine protein kinases (TPK).

Kinases associated with Huntington's disease include, without limitation, mitogen-activated protein kinase, MEK, ERK, JNK, IKK, cell division protein kinase 5 (CDK5), AKT, MKP1.

An exemplary list of kinases useful in the methods of this disclosure include the following: AKT S473; AKT T308; ERK P44; GSK3B S6; GSK3B S9; GSK3 T216; GSK3A S21; MAPK T202; mTOR S2448; mTOR c1/2 T246; mTOR c1/2 S638; JNK 1/2/3; JNK pY183; JNK PY185; MEK 1/2 S217; MEK S221; PI3K p85; PI3K T458; PKB S473; PI3K p55-T199; PKB T308.

These diseases also share in common the accumulation of toxic oligomeric polypeptide species, and in some cases abnormally phosphorylated oligomeric or monomeric forms, and the ability to detect such forms in neuronally derived extracellular vesicles.

### 1. Catalytic Enzymes

A catalytic enzyme can function as a biomarker in a classifier as disclosed herein. Catalytic enzymes involves in neurodegenerative processes are useful in the methods described here. For example, in the case of Parkinson's Disease, enzymes involved in L-DOPA production can function as biomarkers. In particular, one such enzyme is tyrosine hydroxylase (“TH”). Tyrosine hydroxylase (also referred to as tyrosine 3-monooxygenase) is the enzyme responsible for catalyzing the conversion of the amino acid L-tyrosine to L-3,4-dihydroxyphenylalanine (L-DOPA). Catalytic enzymes can be in phosphorylated or unphosphorylated states.

Exemplary catalytic enzymes include TH total, and the phosphorylated forms, TH S40, TH S19 and TH S32.

### C. Neurodegeneration-Associated Proteins

As used herein, the term “neurodegeneration-associated protein” refers to a protein which, especially in an oligomerized form, is associated with neurodegeneration. Neurodegeneration-associated proteins include, without limitation, alpha-synuclein, tau, amyloid beta and huntingtin. Such proteins are prone to aggregation into oligomeric forms.

It is believed that certain oligomerized forms (and size ranges) or abnormally phosphorylated forms of brain polypeptides underlie a variety of neurodegenerative conditions. This includes, for example, the roles of alpha-synuclein in synucleinopathic conditions, amyloid beta in amyloidopathic conditions, tau in tauopathic conditions and huntingtin in Huntington's disease. In particular, current evidence suggests that α-synuclein oligomers can act as a toxic species in PD and other synucleinopathies. In certain embodiments, the oligomeric species detected is an abnormally phosphorylated species.

Forms of neurodegeneration-associated proteins include, without limitation, (I) at least one oligomeric form; (II) a plurality of oligomeric forms in combination (e.g., all oligomeric forms or a subset of oligomeric forms measured together, e.g., alpha synuclein 2-14 or >4-mers), (III) each of a plurality of different oligomeric forms; (IV) at least one oligomeric form and at least one monomeric form; (V) a plurality of oligomeric forms and at least one monomeric form; and (VI) at least one oligomeric form and a plurality of monomeric forms. Forms of neurodegeneration-associated proteins can be used in models to infer, among other things, neurodegenerative conditions or progression toward neurodegenerative conditions, typically with one or more oligomeric forms included in a model indicating the presence and activity of the disease or progression towards the disease. This includes increasing relative amounts of oligomeric alpha-synuclein forms indicating the presence and activity of a synucleinopathy, or progression towards a synucleinopathy; increasing relative amounts of oligomeric amyloid beta indicating the presence and activity of an amyloidopathy, or progression towards an amyloidopathy, increasing relative amounts of oligomeric or abnormally phosphorylated tau indicating the presence and activity of a tauopathy, or progression towards a tauopathy, and increasing relative amounts of oligomeric huntingtin indicating the presence and activity of Huntington's disease, or progression towards Huntington's disease. Accordingly, an abnormal profile of such oligomers indicates a process of neurodegeneration.

Neurodegeneration-associated proteins forms can include one or more oligomeric forms and, optionally, one or more monomeric forms. This includes amounts of species of oligomeric and, optionally, monomeric alpha-synuclein; oligomeric and, optionally, monomeric amyloid beta, oligomeric and, optionally hyperphosphorylated and, optionally, monomeric tau; and oligomeric and, optionally, monomeric huntingtin. For example, a biomarker profile can include (I) at least one oligomeric form; (II) a plurality of oligomeric forms; (III) at least one oligomeric form and at least one monomeric form; (IV) a plurality of oligomeric forms and at least one monomeric form; (V) at least one oligomeric form and a plurality of monomeric forms; and (VI) a plurality of oligomeric forms and a plurality of monomeric forms.

Protein forms can refer to individual protein species or collections of species. For example, a 6-mer of alpha-synuclein is a form of alpha backspace-synuclein. Also, the collection of 6-mers to 18-mers of alpha-synuclein, collectively, can be a form of alpha-synuclein.

A biomarker profile can include a plurality of forms of a protein. In one embodiment, a biomarker profile can include quantitative measures of each of a plurality of oligomeric forms and monomeric form of the neurodegeneration-associated protein. So, for example, the biomarker profile could include quantitative measures of each of a dimer, trimer, tetramer, 5-mer, 6-mer, 7-mer, 8-mer, 9-mer, 10-mer, 11-mer, 12-mer, 13-mer, 14-mer, 15-mer, 16-mer, 19-mer, 20-mer, 24-mer, 50-mer, etc.

As used herein, a “synuclein biomarker profile” refers to a profile comprising oligomeric and, optionally, monomeric alpha-synuclein, the term “amyloid biomarker profile” refers to a profile comprising oligomeric and, optionally, monomeric beta-amyloid, the term “tau biomarker profile” refers to a profile comprising oligomeric and, optionally, monomeric tau, the term “huntingtin biomarker profile” refers to a profile comprising oligomeric and, optionally, monomeric huntingtin.

As used herein, the term “monomeric protein/polypeptide” refers to a single, non-aggregated protein or polypeptide molecule, including any species thereof, such as phosphorylated species. As used herein, the term “oligomeric protein/polypeptide” refers to individual oligomeric species or an aggregate comprising a plurality of oligomeric species, including phosphorylated species. It is understood that measurement of an oligomeric form of a protein, as used herein, can refer to measurement of all oligomeric forms (total oligomeric form) or specified oligomeric forms. Specified oligomeric forms can include, for example, forms within a particular size range or physical condition such as for example soluble fibrils.

In each of these conditions, it is believed that oligomerized/aggregated forms of polypeptides described herein are toxic to neurons in that the biomarker profiles comprising oligomeric forms and, optionally, monomeric forms of these polypeptides function in models to infer pathologic activity. In particular, increased relative amounts of oligomeric forms as compared with monomeric forms indicate pathology. Measures of these biomarkers can be used to track subject responses to therapies that are either in existence or in development as well as to predict development of disease or the state or progress of existing disease.

D. miRNA

MicroRNAs (“miRNA”) are short, single-stranded RNA molecules of about 22 nucleotides. miRNA hybridize with mRNA molecules to silence them. This can result by cleavage of the mRNA, destabilization of the mRNA through shortening of its poly(A) tail, and decreased efficiency of mRNA translation. miRNA can be identified by isolation and sequencing of RNA molecules in a sample. MicroRNAs useful as biomarkers in the methods described herein include, without limitation, miR-15b-5p, miRNA-24, and miR-27a-3pm m204-5p, 124-3p, and 22-3p.

An exemplary list of miRNAs useful in the methods of this disclosure include: 7-5p; 15b-5p; 19b; 22-3p; 24; 27a-3p 24; 29a; 30c-2-3p; 494-3p; 92b-3p; 106b-3p; 122-5p; 124-3p; 122-5p; 132-3p; 138-5p; 142-3p; 146a-5p; 204-5p; 220-3p; 331-5p; 338-3p; 431-5p; 584-5p; 942-5p; 1468-5p.

### II. Neurodegenerative Conditions and Associated Proteins

### A. Synucleinopathies

### 1. Conditions

As used herein, the terms “synucleinopathy” and “synucleinopathic condition” refer to a condition characterized by abnormal profiles of oligomeric alpha-synuclein, which is an abnormal, aggregated form of alpha-synuclein. In certain embodiments, synucleinopathies manifest as clinically evident synucleinopathic disease such as, for example, PD, Lewy body dementia, multiple system atrophy and some forms of Alzheimer's disease, as well as other rare neurodegenerative disorders such as various neuroaxonal dystrophies. Signs and, optionally, symptoms sufficient for a clinical diagnosis of a synucleinopathic disease are those generally sufficient for a person skilled in the art of diagnosing such conditions to make such a clinical diagnosis.

Parkinson's disease (“PD”) is a progressive disorder of the central nervous system (CNS) with a prevalence of 1% to 2% in the adult population over 60 years of age. PD is characterized by motor symptoms, including tremor, rigidity, postural instability and slowness of voluntary movement. The cause of the idiopathic form of the disease, which constitutes more than 90% of total PD cases, remains elusive, but is now considered to involve both environmental and genetic factors. Motor symptoms are clearly related to a progressive degeneration of dopamine-producing neurons in the substantia nigra. More recently, PD has become recognized one of a group of multi-system disorders, which mainly affect the basal ganglia (e.g., PD), or the cerebral cortex (e.g., Lewy body dementia), or the basal ganglia, brain stem and spinal cord (e.g., multiple system atrophy) and which are all linked by the presence of intracellular deposits (Lewy bodies) consisting mainly of a brain protein called alpha-synuclein. Accordingly, these disorders, along with Hallevorden-Spatz syndrome, neuronal axonal dystrophy, and traumatic brain injury have often been termed “Synucleinopathies”.

Signs and symptoms of PD may include, for example, tremors at rest, rigidity, bradykinesia, postural instability and a festinating parkinsonian gate. One sign of PD is a positive response in these motor dysfunctions to carbidopa-levodopa.

Clinically recognized stages of Parkinson's disease include the following: Stage 1—mild; Stage 2—moderate; Stage 3—middle stage; Stage 4—severe; Stage 5—advanced.

Pramipexole (sold under the brand name Mirapex™) is a drug that is used to treat idiopathic Parkinsonism. Pramipexole has activity as an extracellular signal-regulated kinase (ERK) agonist. Accordingly, determining the effect of pramipexole, and other kinase modulators, on kinase activity is useful in determining effectiveness of the drug on Parkinson's Disease.

At present, the diagnosis of PD mainly rests on the results of a physical examination that is often quantified by the use of the modified Hoehn and Yahr staging scale (Hoehn and Yahr, 1967, Neurology, 17:5, 427-442) and the Unified Parkinson's Disease Rating Scale (UPDRS). The differential diagnosis of PD vs. other forms of parkinsonism, e.g., progressive supranuclear palsy (PSP), can prove difficult and misdiagnosis can thus occur in up to 25% of patients. Indeed, PD generally remains undetected for years before the initial clinical diagnosis can be made. When this happens, the loss of dopamine neurons in the substantia nigra already exceeds 50% and may approach 70%. No blood test for PD or any related synucleinopathy has yet been validated. While imaging studies using positron emission tomography (PET) or MRI have been used in the diagnosis of PD by providing information about the location and extent of the neurodegenerative process, they confer little or no information about the pathogenesis of the observed degeneration and do not guide the selection of a particular synucleinopathic-specific intervention.

Lewy body dementias (LBD) affect about 1.3 million people in the US. Symptoms include, for example, dementia, cognitive fluctuations, parkinsonism, sleep disturbances and hallucinations. It is the second most common form of dementia after Alzheimer's disease and usually develops after the age of 50. Like Parkinson's disease, LBD is characterized by abnormal deposits of alpha-synuclein in the brain.

Multiple system atrophy (MSA) is classified into two types, Parkinsonian type and cerebellar type. The parkinsonian type is characterized by, for example, parkinsonian symptoms of PD. The cerebellar type is characterized by, for example, impaired movement and coordination, dysarthria, visual disturbances and dysphagia. MSA symptoms reflect cell loss and gliosis or a proliferation of astrocytes in damaged areas of brain, especially the substantia nigra, striatum, inferior olivary nucleus, and cerebellum. Abnormal alpha-synuclein deposits are characteristic.

Diagnostic error rates for PD and other synucleinopathies can be relatively high, especially at their initial stages, a situation that could become important with the introduction of effective disease modifying therapies, such as neuroprotective therapies.

### 2. Alpha-Synuclein

Alpha-synuclein is a protein found in the human brain. The human alpha-synuclein protein is made of 140 amino acids and is encoded by the SNCA gene (also called PARK1). (Alpha-synuclein: Gene ID: 6622; Homo sapiens; Cytogenetic Location: 4q22.1.)

As used herein, the term “alpha-synuclein” includes normal (unmodified) species, as well as modified species. Alpha-synuclein can exist in monomeric or aggregated forms. Alpha-synuclein monomers can aberrantly aggregate into oligomers, and oligomeric alpha-synuclein can aggregate into fibrils. Fibrils can further aggregate to form intracellular deposits called Lewy bodies. It is believed that monomeric alpha-synuclein and its various oligomers exist in equilibrium. Alpha-synuclein processing in brain can also produce other putatively abnormal species, such as alpha-synuclein phosphorylated at serine 129 (“p129 alpha-synuclein”).

Alpha-synuclein is abundantly expressed in human central nervous system (CNS) and to a lesser extent in various other organs. In brain, alpha-synuclein is mainly found in neuronal terminals, especially in the cerebral cortex, hippocampus, substantia nigra and cerebellum, where it contributes to the regulation of neurotransmitter release. Under normal circumstances, this soluble monomeric protein tends to form a stably folded tetramer that resists aggregation. But, in certain pathological conditions, for unknown reasons, the alpha-synuclein abnormally beta pleats, misfolds, oligomerizes and aggregates to eventually form fibrils, a metabolic pathway capable of yielding highly cytotoxic intermediates.

As used herein, the term “monomeric alpha-synuclein” refers to a single, non-aggregated alpha-synuclein molecule, including any species thereof. As used herein, the term “oligomeric alpha-synuclein” refers to an aggregate comprising a plurality of alpha-synuclein protein molecules. This includes total oligomeric alpha-synuclein and forms or selected species thereof. Oligomeric alpha-synuclein includes forms having at least two monomeric units up to protofibril forms. This includes oligomeric forms having, e.g., between 2 and about 100 monomeric units, e.g., between 4 and 16 monomeric units or at least 2, 3, 4 or 5 dozen monomeric units. As used herein, the term “relatively low weight synuclein oligomer” refers to synuclein oligomers comprised of up to 30 monomeric units (30-mers). Typically, relatively low weight synuclein oligomers are soluble. Accordingly “soluble oligomeric forms” of alpha-synuclein includes oligomers in the size range of 2-mers to 30-mers, e.g., 4-mers to 18-mers. In certain embodiments, alpha-synuclein refers to the form or forms detected by the particular method of detection. For example, the forms can be those detectable with antibodies raised against particular monomeric or oligomeric forms of alpha-synuclein.

The neurotoxic potential of the aberrantly processed alpha-synuclein into oligomerized forms is now believed to contribute to the onset and subsequent progression of symptoms of the aforementioned pathological conditions, notably PD, Lewy body dementia, multiple system atrophy, and several other disorders. These are generally defined as a group of neurodegenerative disorders characterized in part by the intracellular accumulation of abnormal alpha-synuclein aggregates, some of which appear toxic and may contribute to the pathogenesis of the aforementioned disorders. Precisely how certain oligomerized forms of alpha-synuclein might cause neurodegeneration is not yet known, although a role for such factors as oxidative stress, mitochondrial injury, and pore formation has been suggested. Nevertheless, many now believe that processes leading to alpha-synuclein oligomerization and aggregation may be central to the cellular injury and destruction occurring in these disorders.

Some studies have shown that prefibrillar synuclein oligomers and protofibrils are especially prone to confer neurotoxicity (Loov et al., “α-Synuclein in Extracellular Vesicles: Functional Implications and Diagnostic Opportunities”, M. Cell Mol Neurobiol. 2016 April; 36 (3): 437-48. doi: 10.1007/s10571-015-0317-0.) Others suggest that lower order oligomeric synuclein species may be primarily responsible, and it remains hardly clear precisely which synuclein species, or which ensemble of species with differing beta-sheet arrangements, acting alone or in concert by a single or multiple pathologic mechanisms, is most neurotoxic in PD or in any related synucleinopathy (Wong et al., “α-synuclein toxicity in neurodegeneration: mechanism and therapeutic strategies”, Nat Med. 2017 Feb. 7; 23 (2): 1-13. doi: 10.1038/nm.4269).

A portion of intracellular synuclein, along with certain of its metabolic products, is packaged within exosomal vesicles and released into the intracellular fluid in brain from where it passes into the cerebrospinal fluid (CSF) and peripheral blood circulation. Alpha-synuclein is a protein found in the human brain. The human alpha-synuclein protein is made of 140 amino acids and is encoded by the SNCA gene (also called PARK1). (Alpha-synuclein: Gene ID: 6622; Homo sapiens; Cytogenetic Location: 4q22.1.)

### B. Amyloidopathies

### 1. Conditions

As used herein, the term “amyloidopathy” refers to a condition characterized by accumulation of amyloid polymers in the brain. Amyloidopathies include, without limitation, Alzheimer's disease and certain other neurodegenerative disorders such as late stage PD. Alzheimer's Disease is the most prevalent form of dementia. It is characterized at an anatomical level by the accumulation of amyloid plaques made of aggregated forms of beta-amyloid, as well as neurofibrillary tangles. Symptomatically is characterized by progressive memory loss, cognitive decline and neurobehavioral changes. Alzheimer's is progressive and currently there is no known way to halt or reverse the disease.

### 2. Amyloid Beta

Amyloid beta (also called amyloid-β, Aβ, A-beta and beta-amyloid) is a peptide fragment of amyloid precursor protein. Amyloid beta typically has between 36 and 43 amino acids. Amyloid beta aggregates to form soluble oligomers which may exist in several forms. It is believed that misfolded oligomers of amyloid beta can cause other amyloid beta molecules to assume a mis-folded oligomeric form. A-beta₁₋₄₂ has the amino acid sequence: DAEFRHDSGY EVHHQKLVFF AEDVGSNKGA IIGLMVGGVV IA [SEQ ID NO: 1].

In Alzheimer's disease, amyloid-β and tau proteins become oligomerized and accumulate in brain tissue where they have appear to cause neuronal injury and loss; indeed, some aver that such soluble intermediates of aggregation, or oligomers, are the key species that mediate toxicity and underlie seeding and spreading in disease (The Amyloid-β Oligomer Hypothesis: Beginning of the Third Decade. Cline E N, Bicca M A, Viola K L, Klein W L. J Alzheimers Dis. 2018; 64 (s1): S567-S610; “Crucial role of protein oligomerization in the pathogenesis of Alzheimer's and Parkinson's diseases,” Choi M L, Gandhi S. FEBS J. 2018 Jun. 20.) Amyloid β oligomers are crucial for the onset and progression of AD and represent a popular drug target, being presumably the most direct biomarker. Tau protein may also become abnormally hyperphosphorylated.

Methods in current use to quantify monomeric and oligomeric forms of A-beta include enzyme linked immunosorbent assays (ELISA), methods for single oligomer detection, and others, which are mainly biosensor-based methods. (“Methods for the Specific Detection and Quantitation of Amyloid-β Oligomers in Cerebrospinal Fluid”, Schuster J, Funke S A. J Alzheimers Dis. 2016 May 7; 53 (1): 53-67.)

The surface-based fluorescence intensity distribution analysis (sFIDA) features both highly specific and sensitive oligomer quantitation as well as total insensitivity towards monomers (“Advancements of the sFIDA method for oligomer-based diagnostics of neurodegenerative diseases”, Kulawik A. et al., FEBS Lett. 2018 February; 592 (4): 516-534).

### C. Tauopathies

### 1. Conditions

As used herein, the term “tauopathy” refers to a condition characterized by accumulation of and aggregation of in association with neurodegeneration. Tauopathies include, without limitation, Alzheimer's disease (“AD”), progressive supranuclear palsy, corticobasal degeneration, frontotemporal dementia with parkinsonism-linked to chromosome 17, and Pick disease.

AD is also characterized by a second pathological hallmark, the neurofibrillary tangle (NFT). NFTs are anatomically associated with neuronal loss, linking the process of NFT formation to neuronal injury and brain dysfunction. The main component of the NFT is a hyperphosphorylated form of tau, a microtubule-associated protein. During NFT formation, tau forms a variety of different aggregation species, including tau oligomers. Increasing evidence indicates that tau oligomer formation precedes the appearance of neurofibrillary tangles and contributes importantly to neuronal loss. (J Alzheimers Dis. 2013; 37 (3): 565-8 “Tauopathies and tau oligomers”, Takashima A.)

Nonfibrillar, soluble multimers appear to be more toxic than neurofibrillary tangles made up of filamentous tau.

In frontotemporal lobe dementia, full-length TAR DNA Binding Protein (“TDP-43”) forms toxic amyloid oligomers that accumulate in frontal brain regions. TDP-43 proteinopathies, which also include amyotrophic lateral sclerosis (ALS), are characterized by inclusion bodies formed by polyubiquitinated and hyperphosphorylated full-length and truncated TDP-43. The recombinant full-length human TDP-43 forms structurally stable, spherical oligomers that share common epitopes with an anti-amyloid oligomer-specific antibody. The TDP-43 oligomers have been found to be neurotoxic both in vitro and in vivo. (Nat Commun. 2014 Sep. 12; 5:4824. Full-length TDP-43 forms toxic amyloid oligomers that are present in frontotemporal lobar dementia-TDP patients). Determination of the presence and abundance of TDP-43 oligomers can be accomplished using a specific TDP-43 amyloid oligomer antibody called TDP-O among different subtypes of FTLD-TDP (“Detection of TDP-43 oligomers in frontotemporal lobar degeneration-TDP”, Kao P F, Ann Neurol. 2015 August; 78 (2): 211-21.)

### 2. Tau

Tau is a phosphoprotein with 79 potential Serine (Ser) and Threonine (Thr) phosphorylation sites on the longest tau isoform. Tau exists in six isoforms, distinguished by their number of binding domains. Three isoforms have three binding domains and the other three have four binding domains. The isoforms result from alternative splicing in exons 2, 3, and 10 of the tau gene. Tau is encoded by the MAPT gene, which has 11 exons. Haplogroup H1 appears to be associated with increased probability of certain dementias, such as Alzheimer's disease.

Various tau oligomeric species, including those ranging from 6- to 18-mers, have been implicated in the neurotoxic process associated with tauopathic brain disorders and measured by western blot and other techniques including single molecule fluorescence. (See, e.g., Kjaergaard M., et al., “Oligomer Diversity during the Aggregation of the Repeat Region of Tau” ACS Chem Neurosci. 2018 Jul. 17; Ghag G et al., “Soluble tau aggregates, not large fibrils, are the toxic species that display seeding and cross-seeding behavior”, Protein Sci. 2018 Aug. 20. doi: 10.1002/pro.3499; and Comerota M M et al., “Near Infrared Light Treatment Reduces Synaptic Levels of Toxic Tau Oligomers in Two Transgenic Mouse Models of Human Tauopathies”, Mol Neurobiol. 2018 Aug. 17.)

Methods to measure oligomeric tau species include immunoassay. Tau can be isolated by a common expression followed by chromatography, such as affinity, size-exclusion, and anion-exchange chromatography. This form can be used to immunize animals to generate antibodies. Aggregation of tau can be induced using arachidonic acid. Oligomers can be purified by centrifugation over a sucrose step gradient. Oligomeric forms of tau also can be used to immunize animals and generate antibodies. A sandwich enzyme-linked immunosorbent assay that utilizes the tau oligomer-specific TOC1 antibody can be used to detect oligomeric tau.

The tau oligomer complex 1 (TOC1) antibody specifically identifies oligomeric tau species, in the tris insoluble, sarkosyl soluble fraction (Shirafuji N., et al, “Homocysteine Increases Tau Phosphorylation, Truncation and Oligomerization”, Int J Mol Sci. 2018 Mar. 17; 19 (3).) (See, e.g., Methods Cell Biol. 2017; 141:45-64. doi: 10.1016/bs.mcb.2017.06.005. Epub 2017 Jul. 14. Production of recombinant tau oligomers in vitro. Combs B1, Tiernan C T1, Hamel C1, Kanaan N M.)

### D. Huntington's Disease

### 1. Huntington's Disease

Huntington's disease is an inherited disease caused by an autosomal dominant mutation in the huntingtin gene. The mutation is characterized by duplication of CAG triplets. It is characterized by progressive neurodegeneration. Symptoms include movement disorders, such as involuntary movements, impaired gait and difficulty with swallowing and speech. It is also characterized by a progressive cognitive decline.

### 2. Huntingtin Protein

Huntington protein is encoded by the Huntington gene also called HTT or HD. The normal Huntington protein has about 3144 amino acids. The protein is normally about 300 KdA.

In Huntington's disease (HD), cleavage of the full-length mutant huntingtin (mHtt) protein into smaller, soluble aggregation-prone mHtt fragments appears to be a key process in the pathophysiology of this disorder. Indeed, aggregation and cytotoxicity of mutant proteins containing an expanded number of polyglutamine (polyQ) repeats is a hallmark of several diseases, in addition to HD. Within cells, mutant Huntingtin (mHtt) and other polyglutamine expansion mutant proteins exist as monomers, soluble oligomers, and insoluble inclusion bodies. (J Huntingtons Dis. 2012; 1 (1): 119-32. Detection of Mutant Huntingtin Aggregation Conformers and Modulation of SDS-Soluble Fibrillar Oligomers by Small Molecules. Sontag E M, et al., Brain Sci. 2014 Mar. 3; 4 (1): 91-122. Monomeric, oligomeric and polymeric proteins in Huntington disease and other diseases of polyglutamine expansion. Hoffner G. et al.) In certain embodiments, oligomers are 2-10 nm in height with an aspect ratio (longest distance across to shortest distance across) less than 2.5, indicating a globular structure.

### III. Detection and Measurement of Biomarkers

### A. Biological Samples

As used herein, the term “sample” refers to a composition comprising an analyte. A sample can be a raw sample, in which the analyte is mixed with other materials in its native form (e.g., a source material), a fractionated sample, in which an analyte is at least partially enriched, or a purified sample in which the analyte is at least substantially pure. As used herein, the term “biological sample” refers to a sample comprising biological material including, e.g., polypeptides, polynucleotides, polysaccharides, lipids and higher order levels of these materials such as, extracellular vesicles, cells, tissues or organs.

As used herein, the term “extracellular vesicle” refers to membrane bound particles, typically lipid bilayer-delimited, that are naturally released from cells and that have hydrodynamic diameter of from about 50 to about 5000 nm. One example of extracellular vesicles are “exosomes,” which have a diameter of about 50 nm to about 350 nm.

Signaling kinases, as well as forms of neurodegeneration-associated proteins, such as alpha-synuclein, amyloid beta, tau and huntingtin, can be detected in extracellular vesicles from bodily fluid samples from the subject. More particularly, isolates of neuronally derived extracellular vesicles are a preferred subset of extracellular vesicles for the detection and analysis of synucleinopathic conditions. In particular, proteins from internal compartments of an extracellular vesicle are useful.

Extracellular vesicles can be isolated from a variety of biological samples from a subject. In certain embodiments the biological sample is a bodily fluid. Bodily fluid sources of extracellular vesicles include, for example, blood (e.g., whole blood or a fraction thereof such as serum or plasma, e.g., peripheral venous blood), cerebrospinal fluid, saliva, milk and urine, or fractions thereof.

The use of venous blood as a source of extracellular vesicles is a preferred sample for a diagnostic test destined for use in both adults and children due to the safety, acceptability and convenience of routine venipuncture in medical settings. Because target analytes can be present in blood in small amounts, large samples may be taken. For example, a sample can have at least 5 ml, at least 10 ml at least 20 ml of blood. Serum can be prepared by allowing whole blood to clot and removing the clot by, e.g., centrifugation. Plasma can be prepared by, e.g., treating whole blood with an anti-coagulant, such as EDTA, and removal of blood cells by, e.g., centrifugation. The blood sample can be provided by taking the sample from a subject or by receiving the sample from a person who has taken blood from the subject. Blood samples typically will be stored cold, e.g., on ice or frozen at −80° C.

### B. Methods of Measuring Biomarkers

### 1. Signaling Kinases

Kinases convert ATP into ADP in the phosphorylation of substrates. Various assay types to measure kinase activity are known in the art.

### a) Radioactive Scintillation

Radioactive Scintillation assays measure the incorporation of ³²P into a substrate by a kinase.

### b) FRET (Fluorescence Resonance Energy Transfer)

Certain of these assays use amounts of ATP or ADP as indicators of kinase activity. In one such assay, a sample being tested for kinase activity, a substrate for the kinase and ATP are combined. If the kinase is present, it will phosphorylate the substrate using ATP. The remaining ADP can be detected by various assays. One such assay is a FRET (Fluorescence Resonance Energy Transfer) assay in which ADP in the sample after reaction is tagged with one of a donor or acceptor fluorophore. An antibody that binds to ADP and that comprises the other fluorophore of the pair, i.e., an acceptor or donor fluorophore, is added to the mixture. The antibody binds to ADP. Upon excitation, the donor fluorophore transfers energy to the acceptor fluorophore, which fluoresces and can be detected.

### c) Immunodetection

In another assay, a specific kinase can be immunoprecipitated using an antibody specific for the kinase. The precipitated kinase is used in a phosphorylation reaction with a substrate of the kinase. The product of a kinase reaction can be detected by Western blot.

### d) Commercially Available Kinase Assays

Many kinase assays are commercially available. These include, for example, essays available from Promega (Promega.com), which are specific for a number of different kinases. Another example is the Adapta® Universal Kinase Assay System available from Thermo Fisher Scientific (ThermoFisher.com). PerkinElmer™ (PerkinElmer.com) commercializes the LANCE® kinase assay, which uses a fluorescently labeled substrate and a europium-labeled antiphospho antibody to recognize a phosphorylated product, which is detectable through FRET. Samdi Tech, Inc. (SamdiTech.com) commercializes label-free assays that use mass spectrometry.

### 2. Catalytic Enzymes

Catalytic enzymes, such as tyrosine hydroxylase, can be detected by any methods known in the art. This includes, for example, activity assays, ELISA and Western Blot.

3. microRNAs

MicroRNAs (“miRNA”) can be detected, for example, by nucleic acid sequencing methods. These can involve converting the RNA into DNA, and employing standard DNA sequencing technologies. One such method qRT-PCR. Assay results can be expressed as ratios of different miRNAs.

### 4. Neurodegeneration-Associated Proteins

Monomeric and oligomeric forms of proteins can be detected by any methods known in the art including, without limitation, immunoassay (e.g., ELISA), mass spectrometry, size exclusion chromatography, Western blot and fluorescence-based methods (e.g., fluorescence spectroscopy or FRET) and proximity ligation assay.

In a Western blot, proteins in a mixture are separated by electrophoresis. Separated proteins are blotted onto a solid support, such as a nitrocellulose filter, typically by electroblotting. Blotted proteins can be detected either by direct binding with a binding agent against a synuclein oligomers, or by indirect binding in which, for example, the blot is contacted with a labeled primary antibody directed against α-synuclein oligomers, which is allowed to bind with the oligomer. Typically, the blot is washed, to remove unbound antibody. Then, the oligomeric forms are detected using a labeled antibody (typically referred to as a secondary antibody) directed against the primary antibody or a tag attached to the primary antibody.

Labels can include, for example, gold nanoparticles, latex beads, fluorescent molecules, luminescent proteins and enzymes that produce detectable products from a substrate. Tags can include, for example, biotin.

Alternatively, oligomeric species in a mixture can be separated from one another and subsequently detected. Oligomeric species in a mixture can be separated by several methods. In one method, species are separated by electrophoresis. This includes gel electrophoresis. Electrophoresis methods include polyacrylamide gel electrophoresis (“PAGE”) and agarose gel electrophoresis. In one method, native PAGE or blue native PAGE are used. Native PAGE Bis-Tris gels are available from, e.g., ThermoFisher®. In a method called packed-capillary electrophoresis, or “pCE”, arbitrarily wide pores are created by packing nonporous colloidal silica in capillaries. Alternatively, species can be separated by chromatography, such as size exclusion chromatography, liquid chromatography or gas chromatography.

Once separated, specific oligomeric forms of α-synuclein can be differentiated. This can be done without the need for binding agents that specifically bind to a particular oligomeric form because they are already separated and, therefore, distinguishable. A binding agent that binds to α-synuclein oligomers, in general, can be used to detect the forms. Their location on a gel, or time or elution from a column can be used to indicate the particular form detected. For example, larger oligomers typically migrate more slowly in a gel than smaller oligomers.

### a) Alpha-Synuclein

Amounts of monomeric alpha-synuclein and oligomeric alpha-synuclein can be determined individually. Alternatively, total alpha-synuclein in the sample can be measured with either of monomeric alpha-synuclein or oligomeric alpha-synuclein and the amount of the other species can be determined based on the difference.

Monomeric, oligomeric and total alpha-synuclein can be detected by, for example, immunoassay (e.g., ELISA or Western blot, e.g., with chemiluminescent detection), mass spectrometry or size exclusion chromatography. Antibodies against alpha-synuclein are commercially available from, for example, Abcam (Cambridge, MA), ThermoFisher (Waltham, MA) and Santa Cruz Biotechnology (Dallas, TX).

The following references described methods of measuring total alpha-synuclein content. Mollenhauer et al. (Movement Disorders, 32:8 p. 1117 (2017)) describes methods of measuring total alpha-synuclein from bodily fluids. Loov et al. (Cell Mol. Neurobiol., 36:437-448 (2016)) describes use of antibodies to isolate L1CAM positive extracellular vesicles from plasma. Abd-Elhadi et al. (Anal Bioanal Chem. (2016) November; 408 (27): 7669-72016) describes methods of determining total alpha-synuclein levels in human blood cells, CSF, and saliva determined by a lipid-ELISA.

Total alpha-synuclein can be detected in an ELISA using, for example, an anti-human α-syn monoclonal antibody 211 (Santa Cruz Biotechnology, USA) for capture and anti-human α-syn polyclonal antibody FL-140 (Santa Cruz Biotechnology, USA) for detection through a horseradish peroxidase (HRP)-linked chemiluminescence assay. Such an approach avoids detection of monomeric α-synuclein, but does not distinguish between the different multimeric forms.

Monomeric and oligomeric forms of alpha-synuclein can be detected by, for example, immunoassays using antibodies specific for the forms. See, e.g., Williams et al. (“Oligomeric alpha-synuclein and β-amyloid variants as potential biomarkers for Parkinson's and Alzheimer's diseases”, Eur J Neurosci. (2016) January; 43 (1): 3-16) and Majbour et al. (“Oligomeric and phosphorylated alpha-synuclein as potential CSF biomarkers for Parkinson's disease”, Molecular Neurodegeneration (2016) 11:7). El-Agnaf O. et al, (FASEB J. 2016; 20:419-425) described detection of oligomeric forms of alpha-synuclein protein in human plasma as a potential biomarker for PD.

Antibodies against alpha-synuclein monomers and oligomers can be produced by immunizing animals with alpha-synuclein monomers or oligomers. (See, e.g., U.S. Publications 2016/0199522 (Lannfelt et al.), 2012/0191652 (El-Agnaf). Alpha-synuclein oligomers can be prepared by the method of El Agnaf (U.S. 2014/0241987), in which freshly prepared α-synuclein solution was mixed with dopamine at 1:7 molar ratio (α-synuclein:dopamine) and incubated at 37° C. Antibodies against different oligomeric forms of alpha-synuclein are also described in Emadi et al. (“Isolation of a Human Single Chain Antibody Fragment Against Oligomeric α-Synuclein that Inhibits Aggregation and Prevents α-Synuclein-induced Toxicity”, J Mol Biol. 2007; 368:1132-1144. [PubMed: 17391701]) (dimers and tetramers) and Emadi et al. (“Detecting Morphologically Distinct Oligomeric Forms of α-Synuclein”, J Biol Chem. 2009; 284:11048-11058. [PubMed: 19141614]) (trimers and hexamers). Protofibril-binding antibodies are described in, for example, U.S. 2013/0309251 (Nordstrom et al.).

Monomeric alpha-synuclein and can be distinguished from polymeric alpha-synuclein by immunoassay using antibodies that are uniquely recognized by oligomeric forms of synuclein. Another method involves detection of mass differences, e.g., using mass spectrometry. Fluorescent methods can be used. (See, e.g., Sangeeta Nath, et al., “Early Aggregation Steps in α-Synuclein as Measured by FCS and FRET: Evidence for a Contagious Conformational Change” Biophys J. 2010 Apr. 7; 98 (7): 1302-1311, doi: 10.1016/j.bpj.2009.12.4290; and Laura Tosatto et al., “Single-molecule FRET studies on alpha-synuclein oligomerization of Parkinson's disease genetically related mutants”, Scientific Reports 5, December 2015.) Another method involves measuring total alpha synuclein, followed by proteinase K digestion of non-pathological alpha synuclein and detection of remaining alpha synuclein. Another method involves an alpha synuclein proximity ligation assay. Protein ligation assay probes are generated from antibodies raised against the protein(s) of interest, one for each of the proteins involved in the putative interaction, which are conjugated to short oligonucleotides. If the probes bind interacting proteins, the oligonucleotides are sufficiently close to prime an amplification reaction, which can be detected by tagged oligonucleotides and observed as punctate signal, with each punctum representing an interaction. (Roberts R F et al., “Direct visualization of alpha-synuclein oligomers reveals previously undetected pathology in Parkinson's disease brain. Brain”, 2015; 138:1642-1657. doi: 10.1093/brain/awv040, and Nora Bengoa-Vergniory et al., “Alpha-synuclein oligomers: a new hope”, Acta Neuropathol. 2017; 134 (6): 819-838).

The relative amount of oligomeric form of alpha-synuclein to monomers can be expressed as a ratio.

Quantity or amount can be expressed as a signal output from an assay or as an absolute amount after conversion, for example from a standard curve, e.g., in terms of mass per volume.

Alpha-synuclein species in the samples can be further stratified. For example, oligomers species can be divided into lower order oligomers, e.g., 2 to 24 monomeric units, higher order oligomers, e.g., 24 to 100 monomeric units, or protofibrils, etc.

### B) Amyloid Beta

Oligomers and monomers can be distinguished using an enzyme-linked immunosorbent assay (ELISA). This assay resembles a sandwich ELISA. The Aβ monomer contains one epitope, while oligomers contain a plurality these epitopes. Hence, if epitope-overlapping antibodies toward the above unique epitope were used for capturing and detecting antibodies, binding to a specific and unique epitope would generate competition between these two antibodies. In other words, the monomer would be occupied by the capturing or detection antibody but not by both. (“Oligomeric forms of amyloid-β protein in plasma as a potential blood-based biomarker for Alzheimer's disease”, Wang M J et al. Alzheimers Res Ther. 2017 Dec. 15; 9 (1): 98. “Potential fluid biomarkers for pathological brain changes in Alzheimer's disease: Implication for the screening of cognitive frailty”, Ruan Q et al., Mol Med Rep. 2016 October; 14 (4): 3184-98. “Methods for the Specific Detection and Quantitation of Amyloid-β Oligomers in Cerebrospinal Fluid,” Schuster J, Funke S A. J Alzheimers Dis. 2016 May 7; 53 (1): 53-67).

Oligomeric forms of amyloid beta for detection include, e.g. 4-24 mers of amyloid beta.

### c) Tau

Tau oligomers in biological fluids, e.g., CSF, can be measured by ELISA and Western blot analysis using anti-tau oligomer antibodies. (Sengupta U, et al., “Tau oligomers in cerebrospinal fluid in Alzheimer's disease”, Ann Clin Transl Neurol. 2017 April; 4 (4): 226-235.

Oligomers of tau for detection include, e.g., low molecular weight oligomers, e.g., no more than 20-mers, e.g., 3-18 mers. The presence of soluble oligomers in the cerebral spinal fluid can be detected with monoclonal anti-oligomer antibodies with Western blot and Sandwich enzyme-linked immunosorbent assay (sELISA). David, M A et al., “Detection of protein aggregates in brain and cerebrospinal fluid derived from multiple sclerosis patients”, Front Neurol. 2014 Dec. 2; 5:251. Oligomeric forms of tau include hyperphosphorylated forms of oligomeric tau.

### d) Huntingtin

Recent quantification studies have made use of TR-FRET-based immunoassays. One detection method that combines size exclusion chromatography (SEC) and time-resolved fluorescence resonance energy transfer (TR-FRET) allows the resolution and definition of the formation, and aggregation of native soluble mHtt species and insoluble aggregates in brain. “Fragments of HdhQ150 mutant huntingtin form a soluble oligomer pool that declines with aggregate deposition upon aging”, Marcellin D. et al., PLOS One. 2012; 7 (9):e44457.

A variety of published techniques have been used to assay oligomeric huntingtin species including, e.g., Agarose Gel Electrophoresis (AGE) analysis (under either native or mildly denaturing, 0.1% SDS conditions or Blue-Native PAGE under native conditions) which provides a number of immunoreactive oligomers; Anti-huntingtin antibodies differentially recognize specific huntingtin oligomers.

A one-step TR-FRET-based immunoassay has been developed to quantify soluble and aggregated mHtt in cell and tissue homogenates (TR-FRET-based duplex immunoassay reveals an inverse correlation of soluble and aggregated mutant huntingtin in Huntington's disease. Baldo B, et al., chem Biol. 2012 Feb. 24; 19 (2): 264-75).

Time-resolved Förster energy transfer (TR-FRET)-based assays represent high-throughput, homogeneous, sensitive immunoassays widely employed for the quantification of proteins of interest. TR-FRET is extremely sensitive to small distances and can therefore provide conformational information based on detection of exposure and relative position of epitopes present on the target protein as recognized by selective antibodies. We have previously reported TR-FRET assays to quantify HTT proteins based on the use of antibodies specific for different amino-terminal HTT epitopes (Fodale, V. et al., “Polyglutamine- and temperature-dependent conformational rigidity in mutant huntingtin revealed by immunoassays and circular dichroism spectroscopy”, PLOS One. 2014 Dec. 2; 9 (12):e112262. doi: 10.1371/journal.pone.0112262. eCollection 2014.

### C. Isolation of Extracellular Vesicles

Extracellular vesicles are extracellular vesicles that are thought to be released from cells upon fusion of an intermediate endocytic compartment, the multivesicular body (MVB), with the plasma membrane.

Many methods of isolating extracellular vesicles are known in the art. These include, for example, immunoaffinity capture methods, size-based isolation methods, differential ultracentrifugation, extracellular vesicle precipitation, and microfluidic-based isolation techniques. (Loov et al., “α-Synuclein in Extracellular Vesicles: Functional Implications and Diagnostic Opportunities”, M. Cell Mol Neurobiol. 2016 April; 36 (3): 437-48. doi: 10.1007/s10571-015-0317-0).

Amounts of extracellular vesicles in a sample can be determined by any of a number of methods. These include, for example, (a) immunoaffinity capture (IAC), (b) asymmetrical flow field-flow fractionation (AF4), (c) nanoparticle tracking analysis (NTA), (d) dynamic light scattering (DLS), and (e) surface plasmon resonance (SPR). Reprinted with permission from. Immunoaffinity capture (IAC) is the extracellular vesicle capturing technology via immunoaffinity using an indirect isolation method. IAC quantifies extracellular vesicles by analyzing color, fluorescence, or electrochemical signals. Asymmetrical flow field-flow fractionation (AF4) separates and quantifies molecules using field-flow fraction and diffusion. Nanoparticle tracking analysis (NTA) separates and quantifies particles according to their size. NTA uses the rate of Brownian motion to analyze particles. This technique also tracks the concentration and size of extracellular vesicles using a light-scattering technique. Dynamic light scattering (DLS) determines particle size by light scattered by particles that exhibit Brownian motion. Surface plasmon resonance (SPR) is an immunoaffinity-based assay that captures extracellular vesicles with receptors on an SPR sensor surface. Binding changes the optical signals of receptors and their resonance can then be quantified through a light source. In another method, extracellular vesicles can be examined by electron microscopy, e.g., by visualizing at 120 KV in the Zeiss LSM 200 Transmission Electron Microscope.

### 1. Immunoaffinity Capture

Immunoaffinity capture methods use antibodies attached to an extraction moiety to bind extracellular vesicles and separates them from other materials in the sample. A solid support can be, for example, a magnetically attractable extracellular vesicle. Latex immunobeads can be used.

Qiagen describes its exoEasy Maxi Kit as using membrane affinity spin columns to efficiently isolate extracellular vesicles and other extracellular vesicles from serum, plasma, cell culture supernatant and other biological fluids.

### 2. Size-Based Methods

Size-based isolation methods include, for example, size exclusion chromatography and ultrafiltration. In size exclusion chromatography a porous stationary phase is used to separate extracellular vesicles based on size. In ultrafiltration, a porous membrane filter is used two separate extracellular vesicles based on their size or weight.

### 3. Differential Ultracentrifugation

Differential ultracentrifugation involves a series of centrifugation cycles of different centrifugal force and duration to isolate extracellular vesicles based on their density and size differences from other components in a sample. The centrifugal force can be, for example, from ˜100,000 to 120,000×g. Protease inhibitors can be used to prevent protein degradation. A prior cleanup step can be used to remove other large material from the sample.

### 4. Density Gradient Ultracentrifugation

Density gradient ultracentrifugation sorts extracellular vesicles using a gradient medium, such as such as sucrose, Nycodenz (iohexol), and iodixanol. Extracellular vesicles are isolated via ultracentrifugation to the layer in which the density of the gradient media is equal to that of the extracellular vesicles.

### 5. Polymer-Based Methods

Extracellular vesicles can be isolated from solutions of biological materials by altering their solubility or dispersibility. For example, addition of polymers such as polyethylene glycol (PEG), e.g., with a molecular weight of 8000 Da, can be used to precipitate extracellular vesicles from solution.

### 6. Microfluidic-Based Methods

Microfluidics-based methods can be used to isolate extracellular vesicles. These includes, for example, acoustic, electrophoretic and electromagnetic methods. For example, an acoustic nanofilter uses ultrasound standing waves to separate extracellular vesicles in a sample according to their size and density.

### 7. Other Methods

Other methods of isolating neuronally derived extracellular vesicles are described in, for example, Kanninnen, K M et al., “Exosomes as new diagnostic tools in CNS diseases”, Biochimica et Biophysica Acta, 1862 (2016) 403-410.

### 8. Enrichment for Neuronally-Derived Extracellular Vesicles

Neuronally derived extracellular vesicles are extracellular vesicles produced by neurons. Preferably, the object of study is CNS-derived extracellular vesicles, that is, extracellular vesicles produced in the central nervous system, as distinguished from the peripheral nervous system. Methods described herein enrich a biological sample comprising extracellular vesicles for neuronally-derived extracellular vesicles and, by extension, CNS derived extracellular vesicles. A sample that is enriched for neuronally-derived extracellular vesicles has a higher ratio of neuronally-derived extracellular vesicles to non-neuronally-derived exosomes, than a sample of a similar type (e.g., a blood sample) that has not been enriched. So, for example, enrichment can be at least two-fold, at least 5-fold, at least 10-fold at least 50-fold or at least 100-fold over an un-enriched sample. In a sample enriched for neuronally-derived extracellular vesicles, neuronally-derived extracellular vesicles may constitute at least 50%, at least 75%, at least 90% or at least 98% of all extracellular vesicles.

Immunoaffinity methods are useful for isolating neuronally derived extracellular vesicles using brain-specific biomarkers (e.g., neural and glial markers) one such marker is L1CAM. Another marker is KCAM. Other relatively brain-specific proteins can also serve in this capacity. neuronally derived extracellular vesicles are characterized by protein markers associated with the brain, including, for example, KCAM, L1CAM and NCAM and DAT (dopamine transporter). (See, e.g., US 2017/0014450, US 2017/0102397, U.S. Pat. No. 9,958,460). neuronally derived extracellular vesicles can be isolated using affinity capture methods. Such methods include, for example, paramagnetic beads attached to antibodies against specific markers such as L1 CAM. (See, e.g., Shi et al., “Plasma exosomal α-alpha-synuclein is likely CNS derived and increased in Parkinson's disease”, Acta Neuropathol. 2014 November; 128 (5): 639-650.)

In another method, anti-CD 171 can be used to enrich for neuronally-derived exosomes.

Extracellular vesicles from dopamine-producing neurons are characterized by the presence of tyrosine hydroxylase. Samples can be enriched for such extracellular vesicles by immunoaffinity methods that target TH.

### D. Extracellular Vesicle Contents

Many proteins, including kinases, linked to the pathogenesis of human neurodegenerative disease, are generated outside the CNS as well as within the brain, and can become attached to the external surface of extracellular vesicles that pass through the blood brain barrier into the peripheral circulation. Accordingly, in certain embodiments of the methods disclosed herein, an exosomal fraction is treated to remove molecules bound to the exosomal surface. This can be done, for example, by stringent washing procedures, such as with a Phosphate Buffer Solution (PBS). After such processing, the contents of the extracellular vesicle can be processed for the assay.

The scrubbed extracellular vesicles can then be lysed, and their internal contents released for analysis.

### IV. Determining Diagnosis, Stage, Progression, Prognosis and Risk of Developing of Neurodegenerative Conditions

Biomarker profiles comprising amounts of biomarkers in a biological sample selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs, and a change in the profiles over time, indicate presence, severity and direction of neurogenerative conditions of the neurodegenerative type. For example, abnormal ratios, e.g., elevated amounts, of the protein biomarker disclosed herein indicate a process of neurodegeneration. This process, unchecked, can lead to manifest symptoms in synucleinopathic conditions. Accordingly, provided herein are methods of ascertaining in a subject (e.g., in either symptomatic or asymptomatic individuals) a diagnosis, stage, progression, rate, prognosis, drug responsiveness and risk of developing a neurodegenerative condition characterized by the abnormal amounts of one or more biomarker proteins (each, referred to herein as a “neuropathic state”, e.g. “synucleinopathic state”, “amyloidopathic state”, “tauopathic state”, “Huntington's state”).

As used herein, the term “diagnosis” refers to a classification of an individual as having or not having a particular pathogenic condition, including, e.g., the stage of that condition.

As used herein, the term “clinically similar but etiologically different” refers to conditions that share clinical signs and/or symptoms, but which arise from different biological causes.

As used herein, the term “stage” refers to the relative degree of severity of a condition, for example, suspected disease, an early stage, a middle stage or an advanced stage. Staging can be used to group patients based on etiology, pathophysiology, severity, etc.

As used herein, the term “progression” refers to a change, or lack thereof, in stage or severity of a condition over time. This includes an increase, a decrease or stasis in severity of the condition. In certain embodiments, rates of progression, that is, change over time, are measured.

As used herein, the term “prognosis” refers to the predicted course, e.g., the likelihood of progression, of the condition. For example, a prognosis may include a prediction that severity of the condition is likely to increase, decrease or remain the same at some future point in time. In the context of the present disclosure, prognosis can refer to the likelihood that an individual: (1) will develop a neurodegenerative condition, (2) will progress from one stage to another, more advanced, stage of the condition, (3) will exhibit a decrease in severity of the condition, (4) will exhibit functional decline at a certain rate, (5) will survive with a condition for a certain period of time (e.g., survival rate) or (6) will have recurrence of the condition. The condition can be a synucleinopathic condition (e.g., PD, Lewy body dementia, multiple system atrophy or some related synucleinopathy), an amyloidopathic condition (e.g., Alzheimer's disease), a tauopathic condition (e.g., Alzheimer's disease), and Huntington's disease. These terms are not intended to be absolute, as will be appreciated by any one of skill in the field of medical diagnostics.

As used herein, the term “risk of developing” refers to a probability that an individual who is asymptomatic or preclinical will develop to a definitive diagnosis of disease. Determining probability includes both precise and relative probabilities such as “more likely than not”, “highly likely”, “unlikely”, or a percent chance, e.g., “90%”. Risk can be compared with the general population or with a population matched with the subject based on any of age, sex, genetic risk, and environmental risk factors. In such a case, a subject can be determined to be at increased or decreased risk compared with other members of the population. A subject at increased risk of developing a neurodegenerative condition is likely to positively respond to treatment for a neurodegenerative condition, for example, by prevention of developing the condition, delayed onset of the condition or reduced severity of symptoms or morbidity associated with the condition.

### V. Modeling Profiles of Kinases to Infer Diagnosis, Stage, Progression, Prognosis and Risk of Developing of Neurodegenerative Conditions

Determining diagnosis, stage, progression rate, prognosis and risk of a neurodegenerative condition are processes of classifying a subject into different conditions or different classes or conditions within a state, such as disease/health (diagnosis), stage I/stage II/stage III (stage), likely to remiss/likely to progress (prognosis) or assigning a score on a range. Methods of classification using biomarker profiles can involve identifying profiles that are characteristic of various states and correlating a profile from a subject with class or state. Identifying such profiles can involve analysis of biomarker profiles from subjects belonging to different states and discerning patterns or differences between the profiles. Analysis can be done by visual examination of the profiles or by analysis.

### A. Analysis

As used herein, the term “analysis” refers to any algorithm or function that transforms inputs into outputs (e.g., maps inputs to outputs). Analyses include, without limitation, statistical analyses, machine learning analyses and neural net analyses.

Typically, analysis involves analysis of a sufficiently large number of samples to provide statistically meaningful results. Any statistical method known in the art can be used for this purpose. Such methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elasticnet regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state.

Analysis can be operator implemented or implemented by machine learning.

### B. Machine Learning

In certain embodiments analysis is enhanced through the use of machine learning tools. Such tools employ learning algorithms, in which the relevant variable or variables are measured in the different possible states, and patterns differentiating the states are determined and used to classify a test subject. Accordingly, any classification method of this disclosure can be developed by comparing measurements of one or more variables in subjects belonging to the various conditions within a particular synucleinopathic state. This includes, for example, determining a biomarker profile comprising amounts of biomarkers selected from selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs in subjects with various diagnoses or at various stages at various times to allow prediction of diagnosis, stage, progression, prognosis, drug responsiveness or risk. Other variables can be included as well, such as family history, lifestyle, exposure to chemicals, various phenotypic traits, etc.

### 1. Training Dataset

A training dataset is a dataset typically comprising a vector of measures for each of a plurality of features for each of a plurality of subjects (more generally referred to as objects). One of the features can be a classification of the subject, for example, a diagnosis or a measure of a degree on a scale. This can be used in supervised learning methods. Other features can be, for example, measured amounts of biomarkers selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs. So, for example, a vector for an individual subject can include a diagnosis of a neurodegenerative condition (e.g., diagnosed as having or not diagnosed as having Parkinson's Disease) and measurements of each of a plurality of biomarkers as described herein. In certain embodiments, the training dataset used to generate the classifier comprises data from at least 100, at least 200, or at least 400 different subjects. The ratio of subjects classified has having versus not having the condition can be at least 2:1, at least 1:1, or at least 1:2. Alternatively, subjects pre-classified as having the condition can comprise no more than 66%, no more than 50%, no more than 33% or no more than 20% of subjects.

### 2. Learning Algorithms

Learning algorithms, also referred to as machine learning algorithms, are computer-executed algorithms that automate analytical model building, e.g., for clustering, classification or profile recognition. Learning algorithms perform analyses on training datasets provided to the algorithm.

Learning algorithms output a model, also referred to as a classifier, classification algorithm or diagnostic algorithm. Models receive, as input, test data and produce, as output, an inference or a classification of the input data as belonging to one or another class, cluster group or position on a scale, such as diagnosis, stage, prognosis, disease progression, responsiveness to a drug, etc.

A variety of machine learning algorithms can be used to infer a condition or state of a subject. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees), random forests, linear classifiers (e.g., multiple linear regression (MLR), partial least squares (PLS) regression and principal components regression (PCR)), hierarchical clustering and cluster analysis. The learning algorithm will generate a model or classifier that can be used to make an inference, e.g., an inference about a disease state of a subject.

### 3. Validation

A model may be subsequently validation using a validation dataset. Validation datasets typically include data on the same features as the training dataset. The model is executed on the training dataset and the number of true positives, true negatives, false positives and false negatives is determined, as a measure of performance of the model.

The model can then be tested on a validation dataset to determine its usefulness. Typically, a learning algorithm will generate a plurality of models. In certain embodiments, models can be validated based on fidelity to standard clinical measures used to diagnose the condition under consideration. One or more of these can be selected based on its performance characteristics.

c. Model Execution and Making an Inference

The model selected can either result from operator executed analysis or machine learning. In any case, the model can be used to make inferences (e.g., predictions) about a test subject. A biomarker profile, for example in the form of a test dataset, e.g., comprising a vector, containing values of features used by the model, can be generated from a sample taken from the test subject. The test dataset can include all of the same features used in the training dataset, or a subset of these features. The model is then applied to or executed on the test dataset. Correlating a biomarker profile with a condition, disease state, a prognosis, a risk of progression, a likelihood of drug response, etc. is a form of executing a model. Correlating can be performed by a person or by a machine, e.g., by a programmable digital computer. The choice may depend on the complexity of the operation of correlating. This produces an inference, e.g., a classification of a subject as belonging to a class or a cluster group (such as a diagnosis), or a place on a scale (such as likelihood of responding to a therapeutic intervention).

In certain embodiments the classifier will include a plurality of oligomeric protein forms and, typically, but not necessarily, one or more monomeric forms of the neurodegeneration-associated protein. The classifier may or may not be a linear model, e.g., of the form AX+BY+CZ=N, wherein A, B and C are measured amounts of forms X, Y and Z. The classifier may require, for example, support vector machine analysis. For example, the inference model may perform a pattern recognition in which a biomarker profile lies on a scale between normal and abnormal, with various profiles tending more toward normal or toward abnormal. Thus, the classifier may indicate a confidence level that the profile is normal or abnormal. An abnormal biomarker profile can be a profile that, when analyzed by a classification algorithm, classifies a subject into a non-normal category, such as disease being present, or at increased risk of disease. A measure of a biomarker may be abnormal if the measure lies outside a range considered normal, for example, a deviation from a normal range that is statistically significant.

The classifier or model may generate, from the one or are plurality of forms measured, a single diagnostic number which functions as the model. Classifying a neuropathological state, e.g., synucleinopathic state (e.g., diagnosis, stage, progression, prognosis and risk) can involve determining whether the diagnostic number is above or below a threshold (“diagnostic level”). For example, the diagnostic number can be a relative amount of two different signaling kinases. That threshold can be determined, for example, based on a certain deviation of the diagnostic number above normal individuals who are free of any sign of a neurodegenerative, e.g., synucleinopathic, condition. A measure of central tendency, such as mean, median or mode, of diagnostic numbers can be determined in a statistically significant number of normal and abnormal individuals. A cutoff above normal amounts can be selected as a diagnostic level of a neurodegenerative, e.g., synucleinopathic, condition. That number can be, for example, a certain degree of deviation from the measure of central tendency, such as variance or standard deviation. In one embodiment the measure of deviation is a Z score or number of standard deviations from the normal average.

The model can be selected to provide a desired level of sensitivity, specificity or positive predictive power. For example, the diagnostic level can provide a sensitivity of at least any of 80%, 90%, 95% or 98% and/or a specificity of at least any of 80%, 90%, 95% or 98%, and/or a positive predictive value of at least any of 80%, 90%, 95% or 98%. The sensitivity of a test is the percentage of actual positives that test positive. The specificity of a test is the percentage of actual negatives that test negative. The positive predictive value of a test is the probability that a subject that tests positive is an actual positive.

### 1. Exemplary Indices

In one method, the index or classifier is a function of several variables. These variables include biomarkers from two or more groups consisting of (1) one or more enzymes selected from signaling kinases and/or catalytic enzymes, (2) one or more neurodegeneration-associated proteins in monomeric or oligomeric form, and (3) one or more miRNAs. Certain indices use at least one or more signaling kinases and one or more neurodegeneration-associated proteins, in particular, oligomeric forms of a neurodegeneration-associated protein. Other indices use biomarkers from all three groups. Other indices use both one or more signaling kinases and one or more catalytic enzymes. Other indices use one or more signaling kinases, one or more catalytic enzymes and one or more neurodegeneration-associated protein forms. Other indices use one or more signaling kinases, one or more catalytic enzymes, one or more neurodegeneration-associated protein forms and one or more miRNAs.

Other indices use relative amounts (e.g., a ratio) of up-regulated biomarkers to down-regulated biomarkers of the neurodegenerative condition. For example, certain indices use relative amounts of a group comprising one or more up-regulated signaling kinases (e.g., AKT), one or more up-regulated catalytic enzymes (e.g., TH-S40), and one or more up-regulated neurodegeneration-associated protein forms (e.g., alpha synuclein oligomers); and a group comprising one or more down-regulated signaling kinases (e.g., MAPK), one or more down-regulated catalytic enzymes (e.g., TH (total protein)), or relatively upregulated when measured in certain phosphorylated forms, e.g. TH S40 (among others), or relatively down regulated during the administration of dopaminergic drugs for the treatment of PD symptoms. Other indices use relative amounts of a group comprising a plurality of up-regulated signaling kinases (e.g., AKT), one or more synuclein oligomers and one or more miRNA, and a group comprising a plurality of down-regulated signaling kinases (e.g., MAPK) and one or more down-regulated catalytic enzymes (e.g., TH (e.g., total protein).

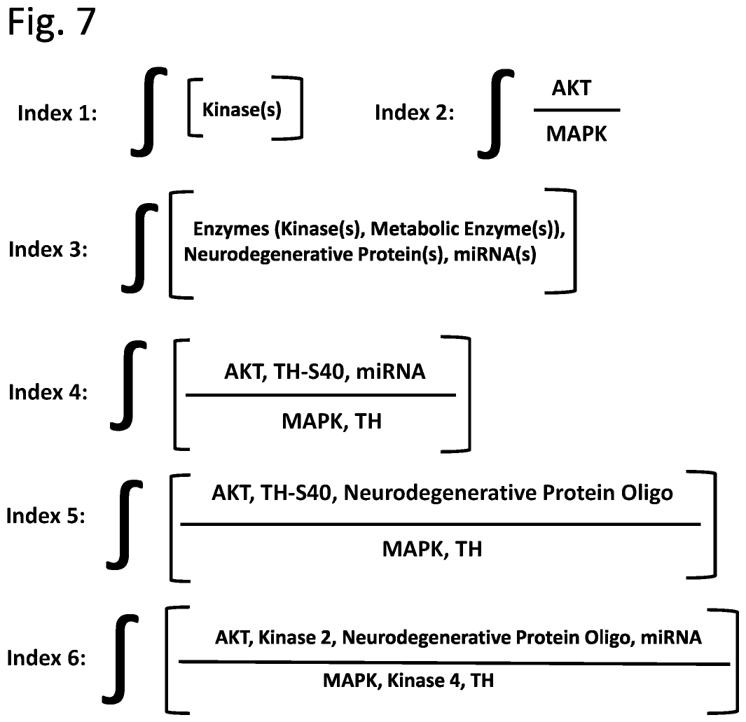

Referring to FIG. 7, this disclosure contemplates a variety of diagnostic indices. Diagnostic indices can use, as biomarker sets, (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs.

A diagnostic index for neurodegenerative disease can be a function of one or more phosphorylated signaling kinases. (FIG. 7, Index 1.) For example, the index can include a plurality of signaling kinases. One such index for Parkinson's disease is a function of relative amounts of AKT and MAPK3. Increases in this index are positively associated with Parkinson's disease. (FIG. 7, Index 2.)

In another index (FIG. 7, Index 3), the diagnostic is a function of one or more signaling kinases, one or more catalytic enzymes, one or more neurodegeneration-associated protein forms and one or more microRNAs.

In another index for Parkinson's Disease, the index is a function of AKT, phosphorylated tyrosine hydroxylase, an miRNA, MAPK3, and a total or non-phosphorylated tyrosine hydroxylase; and, in one version, a function of relative amounts of (AKT, phosphorylated tyrosine hydroxylase, an miRNA) to (MAPK3 and total/non-phosphorylated tyrosine hydroxylase protein). (FIG. 7, Index 4.)

In another index for Parkinson's Disease, the index is a function of AKT, phosphorylated tyrosine hydroxylase, one or more neurodegeneration-associated protein forms, MAPK3, and a non-phosphorylated tyrosine hydroxylase; and, in one version, a function of relative amounts of (AKT, phosphorylated tyrosine hydroxylase, one or more neurodegeneration-associated protein forms) to (MAPK3 and non-phosphorylated tyrosine hydroxylase). (FIG. 7, Index 5.)

In another index for Parkinson's Disease, the diagnostic is a function of AKT, MAPK, optional second and third signaling kinases, one or more catalytic enzymes, one or more neurodegeneration-associated protein forms and one or more microRNAs. In a specific version for Parkinson's Disease, the index is a function of AKT, a second signaling kinase, one or more neurodegeneration-associated proteins, one or more miRNAs, MAPK3, a fourth signaling kinase and a non-phosphorylated tyrosine hydroxylase. In one version, the index is a function of relative amounts of (AKT, a second signaling kinase, one or more neurodegeneration-associated proteins, and one or more miRNAs) to (MAPK3, a fourth signaling kinase and a non-phosphorylated tyrosine hydroxylase). (FIG. 7, Index 6.) AKT can be measured in its phosphorylated forms, such as AKT S473. MAPK3 can be measured in its phosphorylated form MAPK T202.

### VI. Development of Therapeutic Interventions to Treat Neurodegenerative Conditions

In another aspect, provided herein are methods to enable the practical development of therapeutic interventions for neurodegenerative conditions, e.g., synucleinopathic conditions, amyloidopathic conditions, tauopathic conditions, and Huntington's disease. The methods involve, among other things, selecting subjects for clinical trials and determining effectiveness of the therapeutic intervention in a set of subjects.

Methods comprising monitoring the biomarker profiles of neurodegeneration-associated proteins are useful to determine whether an experimental therapeutic intervention is effective in preventing clinical onset or inhibiting subsequent progression of a synucleinopathy, or whether a subject should be entered into a clinical trial to test the efficacy of a drug candidate to treat such conditions. Biomarker profiles or changes in biomarker profiles of the neurodegeneration-associated protein enable the direct determination of treatment effects on the condition, including, e.g., basic disease process.

### A. Subject Enrollment

Clinical trials involve enrollment of subjects for testing the efficacy and safety of a potential therapeutic intervention, such as a pharmaceutical. Typically, while subjects are selected on basis of certain common characteristics, they also manifest important differences in other conditions of a state, e.g., subjects with or without a diagnosis of disease or at different stages of disease or different subtypes of disease or different prognosis. Clinical trial subjects can be stratified into different groups to be treated the same or differently. Stratification can be based on any number of factors, including, stage of disease. Disease Staging is a classification system that uses diagnostic findings to produce clusters of patients based on such factors as etiology, pathophysiology and severity. It can serve as the basis for clustering clinically homogeneous patients to assess quality of care, analysis of clinical outcomes, utilization of resources, and the efficacy of alternative treatments.

In another method, potential clinical trial subjects are stratified at least in part on biomarker profiles. Thus, for example, subjects having different biomarker profiles (e.g., higher and lower relative amounts or rates of change over time) can be assigned to different groups.

The population of subjects in a clinical trial should be sufficient to show whether the drug produces a statistically significant difference in outcome. Depending on this power level, the number of individuals in the trial can be at least 20, at least 100 or at least 500 subjects. Among these, there must be a significant number of individuals exhibiting a biomarker profile consistent with having the neurodegenerative condition (e.g., an increased level of the biomarker. For example, at least 20%, at least 35%, at least 50%, or at least 66% of the subjects may initially have such a biomarker profile (comprising, e.g., various species of signaling kinases). Also, a significant number of subjects are to be divided between class states. For example, at least 20%, at least 35%, at least 50%, at least 66% or 100% of the subjects may initially have a diagnosis of a neurodegenerative condition (e.g., synucleinopathic condition (e.g., PD), amyloidopathic condition, tauopathic condition and Huntington's disease).

### B. Drug Development

Upon commencement of the clinical trial effectiveness of the therapeutic intervention on the different stratification groups can be rapidly determined as a function of the effect on the biomarker profile that includes biomarkers selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs. More specifically, a change in the biomarker profile predicts the clinical effectiveness of the therapeutic intervention. Methods generally involve first testing individuals to determine biomarker profile comprising signaling kinases, and, optionally, neurodegeneration-associated proteins. After the measurements, the therapeutic intervention, e.g., an experimental drug, is administered to at least a subset of the subjects. Typically, at least a subset of the subjects is given a placebo or no treatment. In some cases, subject serve as their own controls, first receiving a placebo, and then, the experimental intervention, or the reverse, for comparison. In certain instances, this can be done in conjunction with administering already recognized forms of treatment. The population can be divided in terms of dosing, timing and rate of administration of the therapeutic intervention. Ethical considerations may require stopping a study when a statistically significant improvement is seen in test subjects. As used herein, “experimental drug” and “drug candidate” refer to an agent having or being tested for a therapeutic effect. A “putative neuroprotective agent” refers to an agent having or being tested to have neuroprotective action.

After administration of the therapeutic intervention, the biomarker profile is determined again, and can be further evaluated as a rate of change function.

The therapeutic intervention can be the administration of a drug candidate. Using standard statistical methods, it can be determined whether the therapeutic intervention has had a meaningful impact on the biomarker profile comprising biomarkers selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs. In general, a statistically significant change, especially a shift toward a more normal profile, compared with the initial biomarker profile indicates that the therapeutic intervention is neuroprotective and thus will delay clinical onset, or slow or preferably reverse progression of the neurodegenerative condition (e.g., synucleinopathic condition, amyloidopathic condition, tauopathic condition, Huntington's disease.

Accordingly, subjects for whom a biomarker profile comprising biomarkers selected from (1) at least one signaling kinase and, optionally, at least one oligomeric form of a neurodegeneration-associated protein, or (2) each of one or a plurality of different signaling kinases can be measured include, for example: (1) Subjects who are asymptomatic for a neurodegenerative condition (e.g., synucleinopathic condition, amyloidopathic condition, tauopathic condition, Huntington's disease); (2) subjects having minimal neurodegenerative disease symptoms and no signs suggestive of a neurodegenerative condition (e.g., who may be diagnosed with “suspected” or “preclinical” for a neurodegenerative condition, especially when certain genetic and/or environmental risk factors have been identified); (3) subjects having the diagnosis of “probable” neurodegenerative condition and subjects diagnosed (“definitive diagnosis”) with a neurodegenerative condition. These include, for example, (1) subjects who are asymptomatic for a synucleinopathic condition, (2) subjects having minimal parkinsonian symptoms and no signs suggestive of a synucleinopathic condition (e.g., who may be diagnosed with “suspected” or “preclinical” for PD or some related synucleinopathy, especially when certain genetic and/or environmental risk factors have been identified); (3) subjects having the diagnosis of “probable” synucleinopathy (e.g., PD) and subjects diagnosed (“definitive diagnosis”) with a synucleinopathic condition.

Subjects are typically human but also include nonhuman animals, for example, those used as models for PD, such as, rodents (e.g., mice and rats), cats, dogs, other domesticated quadrupeds (such as horses, sheep and swine), and nonhuman primates (e.g., monkeys). Animal models include both genetic models and models based on the administration of neurotoxins. Neurotoxins used in such models include, for example, 6-hydroxydopamine (6-OHDA) and 1-methyl-1,2,3,6-tetrahydropyridine (MPTP) administration, and paraquat and rotenone. Genetic models include genetic mutations in SNCA (α-syn, PARK1, and 4), PRKN (parkin RBR E3 ubiquitin protein ligase, PARK2), PINK1 (PTEN-induced putative kinase 1, PARK6), DJ-1 (PARK7), and LRRK2 (leucine-rich repeat kinase 2, PARK8).

Clinical trials for neuroprotective therapies for neurodegenerative conditions, such as synucleinopathies require measures that promptly indicate the effectiveness of the potential therapy. Otherwise, the determination of drug efficacy based on clinical observations of a non-quantitative nature taking many months. Biomarker profiles comprising neurodegeneration-associated protein oligomers and, optionally monomers provide such measures, thus enabling the practical evaluation of disease modifying drug efficacy in subjects suffering from fatal brain disorders such as PD.

### C. Validation

Subjects are said to respond to therapy when they show a clinically significant improvement in clinical symptoms. Efficacy of a drug being tested is typically validated by clinical measurements, for example, by determining disease symptoms, signs and stages. Such clinical measures include those described herein, such as the modified Hoehn and Yahr staging scale and the Unified Parkinson's Disease Rating Scale (UPDRS). Biomarker profiles as described herein also provide an indication of response to therapy and can do so at much earlier time periods than other forms of clinical evaluation. This will typically happen after the drug has been validated using traditional methods. However, biomarker profiles can be used in addition to or instead of clinical markers to determine efficacy of a drug in a subject or a population of subjects. For example, responses that can be detected by traditional means only about 18 months after initiation of therapy can be detected in biomarker profiles at as little as 12 months, six months or three months after initiation of therapy. Accordingly, in some embodiments determination of response to therapy involves determining a first biomarker profile of the subject at a first time point, administering a therapeutic intervention to the subject; determining a second biomarker profile after administration of the therapeutic intervention e.g., within about any of one month, three months, six months, nine months, 12 months, 15 months, or 18 months of initiation of therapy; and comparing the first and second biomarker profiles to identify changes. No statistically significant difference in the biomarker profiles indicates no response to therapy. A statistically significant change toward a normal biomarker profile indicates a positive response to therapy while a statistically significant change away from a normal profile indicates a negative response to therapy or, progression of the disease. Where a normal profile is known before the therapeutic intervention is initiated, measurement of the first biomarker profile can be dispensed with and the determination can rely on the second biomarker profile.

Early evidence, based on biomarker analysis, of a lack of efficacy of a therapeutic intervention can be used to halt administration of the therapeutic intervention. with its attendant risks, thus improving the safety of trial participants.

### VII. Methods of Treatment

Depending on the stage or class of neurodegenerative condition (e.g., synucleinopathic condition, amyloidopathic condition, tauopathic condition, Huntington's disease) into which a subject is classified based on the biomarker profile as described herein, a subject may be in need of a therapeutic intervention. Provided herein are methods of treating a subject determined, by the methods disclosed herein, to exhibit a neurodegenerative condition (e.g., a synucleinopathic condition, and amyloidopathic condition, a tauopathic condition, Huntington's disease) with a therapeutic intervention effective to treat the condition. Therapeutic interventions that change and especially those that return levels of signaling kinases and, optionally, neurodegeneration-associated proteins, reflect an effective treatment, e.g., a therapeutic intervention developed by the methods herein, and clinically validated.

As used herein, the terms “therapeutic intervention”, “therapy” and “treatment” refer to an intervention that produces a therapeutic or beneficial effect, (e.g., is “therapeutically effective”). Therapeutically effective interventions prevent, slow the progression of, delay the onset of symptoms of, improve the condition of (e.g., causes remission of), improve symptoms of, or improve the quality of life of, or prolong the life of, or cure a disease, such as a synucleinopathic condition. A therapeutic intervention can include, for example, administration of a treatment, administration of a pharmaceutical, or a biologic or nutraceutical substance with therapeutic intent. A therapeutic intervention is typically administered by a medical care professional, e.g., a physician or nurse. The response to a therapeutic intervention can be complete or partial. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. Recognizing that certain sub-groups of subjects may not respond to a therapy, one measure of therapeutic effectiveness can be effectiveness for at least 90% of subjects undergoing the intervention over at least 100 subjects.

As used herein, the term “effective” as modifying a therapeutic intervention (“effective treatment” or “treatment effective to”) or amount of a pharmaceutical drug (“effective amount”), refers to that treatment or amount to ameliorate a disorder, as described above. For example, for the given parameter, a therapeutically effective amount will show an increase or decrease in the parameter of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. Currently, clinically efficacy against the severity of motor symptoms in a parkinsonian subject can be measured using such standardized scales as the UPDRS and the Hoehn and Yahr scale; for metal and cognitive symptoms the ADAS-cog or the MMPI scales. (It is recognized that the utility of such scales does not necessarily depend on the type or nature of the underlying disease condition.)

Thus, according to some methods a subject is first tested for the biomarker profile comprising forms of oligomeric and/or monomeric forms of neurodegeneration-associated proteins in a biological sample from the subject. A classification into an appropriate condition or class is determined based on the biomarker profile. Based on the classification a decision can be made regarding the type, amount, route and timing of administering an optimally effective therapeutic intervention to the subject.

### A. Synucleinopathic Condition

In certain embodiments, a symptom modifying therapeutic intervention for PD (i.e., a symptomatic or palliative treatment) comprises administration of a drug selected from a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)) or some similar agent or group of agents.

In another embodiment, the drug is a combination of a NK1-antagonist and 6-propylamino-4,5,6,7-tetrahydro-1,3-benzothiazole-2-amine. For example, the NK1-antagonist can be rolapitant or aprepitant and the 6-propylamino-4,5,6,7-tetrahydro-1,3-benzothiazole-2-amine is pramipexole dihydrochloride monohydrate. For example, the daily dose of aprepitant can be between 10 mg to 250 mg, and the daily dose of pramipexole dihydrochloride monohydrate can be between from 1.5 mg to 45 mg. (See, e.g., U.S. patent application 2020/0147097. In another embodiment, the drug is combination product comprising delivery of a 5HT3-antagonist in combination with a therapeutically effective daily dose of a 6-propylamino-4,5,6,7-tetrahydro-1,3-benzothiazole-2-amine, e.g., a combination of ondansetron hydrochloride dihydrate and pramipexole dihydrochloride monohydrate. The daily dose of ondansetron hydrochloride dihydrate can be 4 mg to 32 mg and the daily dose of pramipexole can be 1.5 mg to 42 mg. (See, e.g., U.S. Pat. No. 10,799,484.) In certain embodiments, a neuroprotective or disease modifying therapeutic intervention for PD comprises administration of a putatively disease modifying drug as described in any of the following provisional patent applications, incorporated herein by reference in their entirety: Ser. No. 62/477,187, filed Mar. 27, 2017; Ser. No. 62/483,555, filed Apr. 10, 2017; Ser. No. 62/485,082, filed Apr. 13, 2017; Ser. No. 62/511,424, filed May 26, 2017; Ser. No. 62/528,228, filed Jul. 3, 2017; Ser. No. 62/489,016, filed Apr. 24, 2017; Ser. No. 62/527,215, filed Jun. 30, 2017.

### B. Amyloidopathic Condition

In certain embodiments, a symptom modifying therapeutic intervention for an amyloidopathic condition (i.e., a symptomatic or palliative treatment) comprises administration of a drug such as Razadyne® (galantamine), Exelon® (rivastigmine), and Aricept® (donepezil).

### C. Tauopathic Condition

In certain embodiments, a symptom modifying therapeutic intervention for a tauopathic condition (i.e., a symptomatic or palliative treatment) comprises administration of a drug such as Razadyne® (galantamine), Exelon® (rivastigmine), and Aricept® (donepezil) or those cited herein used for the symptomatic treatment of PD.

### D. Huntington's Disease

In certain embodiments, a symptom modifying therapeutic intervention for Huntington's disease (i.e., a symptomatic or palliative treatment) comprises administration of a drug such as tetrabenazine (Austedo® (deutetrabenazine), IONIS-HTT_{Rx}, as well as various neuroleptics and benzodiazepines.

### VIII. Methods of Evaluating Responsiveness to Therapeutic Interventions

In a subject suffering from a neurodegenerative disorder (e.g., a synucleinopathic condition, an amyloidopathic condition, a tauopathic condition, Huntington's disease) the effectiveness of a therapeutic intervention or the responsiveness of the subject to the therapeutic intervention can be determined by assessing the effect of the therapeutic intervention on the biomarker profile. This includes effectiveness in any neurodegenerative state, e.g., diagnosis, stage, progression, prognosis and risk. A change in the biomarker profile toward a more normal profile indicates effectiveness of the therapeutic intervention.

Use of biomarker profiles comprising a set of biomarkers selected from (i) a plurality of different signaling kinases; or (ii) biomarkers from at least two groups selected from: (1) one or more enzymes selected from phosphorylated signaling kinases and/or catalytic enzymes, (2) one or more neurodegeneration-associated proteins in monomeric or oligomeric form, and (3) one or more miRNAs, confers advantages over conventional means (e.g., changes in symptomatology, functional scales or radiologic scans) for judging treatment efficacy in such situations. Not only are such conventional means of judging efficacy insensitive, inexact and semi-quantitative, but typically require long periods (e.g., years) before becoming of sufficient magnitude to accurately measure. Accordingly, the number of potentially useful treatments tested is significantly reduced, and the expense of clinical trials and thus the eventual cost of useful medications is substantially increased.

In certain embodiments, the biomarker profile of the protein biomarker species are measured a plurality of times, typically, before, during and after administration of the therapeutic intervention or at a plurality of time points after the therapeutic intervention.

### IX. Kits

In another aspect, provided herein are kits for detecting biomarkers as described herein. The kits can comprise containers to hold reagents for isolating extracellular vesicles from a bodily fluid, reagents for preferentially isolated neuronally-derived extracellular vesicles, e.g., dopamine neurons, from all extracellular vesicles, and reagents sufficient to detect the kinases and/or forms of neurodegeneration-associated proteins.

For example, kits for use in detecting and staging biomarkers in a biological sample for a neurodegenerative condition can comprise reagents, buffers, enzymes, antibodies and other compositions specific for this purpose. For example, the kit can include containers with antibodies that are specific for a signaling kinase, a catalytic enzyme or a form of a neurodegeneration-associated protein. The kit also can contain chromatographic media for isolating nucleic acid molecules, such as miRNA. Kits can also typically include instructions for use as well as and software for data analysis and interpretation. The kit may further comprise samples that serve as normative standards. Each solution or composition may be contained in a vial or bottle and all vials held in close confinement in a box for commercial sale.

### X. Computer Systems

Databases and operations on them as provided herein can be executed by programmable digital computer.

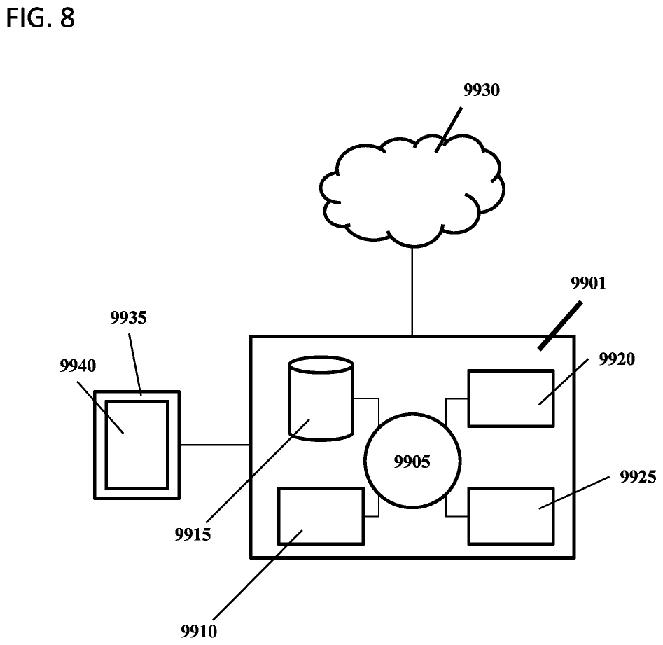

FIG. 8 shows an exemplary computer system. The computer system 9901 includes a central processing unit (CPU, also “processor” and “computer processor” herein) 9905, which can be a single core or multi core processor, or a plurality of processors for parallel processing. The computer system 9901 also includes memory or memory location 9910 (e.g., random-access memory, read-only memory, flash memory), electronic storage unit 9915 (e.g., hard disk), communication interface 9920 (e.g., network adapter) for communicating with one or more other systems, and peripheral devices 9925, such as cache, other memory, data storage and/or electronic display adapters. The computer readable memory 9910, storage unit 9915, interface 9920 and peripheral devices 9925 are in communication with the CPU 9905 through a communication bus (solid lines), such as a motherboard. The storage unit 9915 can be a data storage unit (or data repository) for storing data. The computer system 9901 can be operatively coupled to a computer network (“network”) 9930 with the aid of the communication interface 9920. The network 9930 can be the Internet, an internet and/or extranet, or an intranet and/or extranet that is in communication with the Internet. The network 9930 in some cases is a telecommunication and/or data network. The network 9930 can include one or more computer servers, which can enable distributed computing, such as cloud computing. The CPU 9905 can execute a sequence of machine-readable instructions, which can be embodied in a program or software. The instructions may be stored in a memory location, such as the computer readable memory 9910. The instructions can be directed to the CPU 9905, which can subsequently program or otherwise configure the CPU 9905 to implement methods of the present disclosure.

The storage unit 9915 can store files, such as drivers, libraries and saved programs. The storage unit 9915 can store user data, e.g., user preferences and user programs. The computer system 9901 in some cases can include one or more additional data storage units that are external to the computer system 9901, such as located on a remote server that is in communication with the computer system 9901 through an intranet or the Internet.

The computer system 9901 can communicate with one or more remote computer systems through the network 9930.

Methods as described herein can be implemented by way of machine (e.g., computer processor) executable code stored on an electronic storage location of the computer system 9901, such as, for example, on the computer readable memory 9910 or electronic storage unit 9915. The machine executable or machine-readable code can be provided in the form of software. During use, the code can be executed by the processor 9905 using computer logic (e.g., designed into digital circuits). The code can embody a function or model. In some cases, the code can be retrieved from the storage unit 9915 and stored on the memory 9910 for ready access by the processor 9905. The code can access from memory data in electronic form received and stored. In some situations, the electronic storage unit 9915 can be precluded, and machine-executable instructions are stored on memory 9910.

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system 9901 can include or be in communication with an electronic display 9935 that comprises a user interface (UI) 9940 for providing, for example, input parameters for methods described herein. Examples of UIs include, without limitation, a graphical user interface (GUI) and web-based user interface.

Processes described here can be performed using one or more computer systems that can be networked together. Calculations can be performed in a cloud computing system in which data on the host computer is communicated through the communications network to a cloud computer that performs computations and that communicates, or outputs results to a user through a communications network. For example, an output can be transmitted to a cloud computing system where a utility score algorithm performs one or more operations of the methods described herein. At any step cloud computing system can transmit results of calculations back to the computer operated by the user.

Data can be transmitted electronically, e.g., over the Internet. The data can be output to a device accessible by user. Electronic communication can be, for example, over any communications network include, for example, a high-speed transmission network including, without limitation, Digital Subscriber Line (DSL), Cable Modem, Fiber, Wireless, Satellite and Broadband over Powerlines (BPL). Information can be transmitted to a modem for transmission, e.g., wireless or wired transmission, to a computer such as a desktop computer. Alternatively, reports can be transmitted to a mobile device. Reports may be accessible through a subscription program in which a user accesses a website which displays the report. Reports can be transmitted to a user interface device accessible by the user. The user interface device could be, for example, a personal computer, a laptop, a smart phone or a wearable device, e.g., a watch, for example worn on the wrist.

### EXAMPLES

The following examples are offered by way of illustration and not by way of limitation.

### I. Example 1: Biomarker Profiles are Changed in Synucleinopathic Conditions

A cohort of individuals who are the subject of study have been diagnosed with a synucleinopathic condition. The subjects are given an active therapeutic intervention and then one that is different, possibly known to be inactive. Alternatively, the interventions can be given in the reverse order. Or a cohort comprising a plurality of subjects who are asymptomatic for a synucleinopathic condition in a plurality of subjects who have been diagnosed with the synucleinopathic condition are the subject of study. In either case, venous blood samples are is taken from each subject by venipuncture at various times, including under baseline or control (e.g., inactive intervention treatment) conditions and again during the administration of a potentially active (e.g., experimental intervention) treatment. neuronally derived extracellular vesicles are isolated from the blood using methods described herein. Amounts of biomarkers including (1) one or more enzymes selected from phosphorylated signaling kinases and/or catalytic enzymes, (2) one or more neurodegeneration-associated proteins in monomeric or oligomeric form, and (3) one or more miRNAs that are contained within the isolated extracellular vesicles (e.g., exosomes) are measured. These data are combined into a dataset. The dataset is analyzed using statistical methods, in this case, used to train a learning algorithm, e.g., a support vector machine, to develop a model that infers whether a subject should be classified as having or not having the synucleinopathic condition. Results show that in the cohort of subjects diagnosed with the synucleinopathic condition certain species of signaling kinases have different activity to a statistically significant degree relative to other signaling kinases. Also, oligomeric forms of neurodegeneration-associated proteins also are changed to a statistically significant degree. Those found to have a significant change in the results of this biomarker assay are later found to have a proportional change in clinical state.

### II. Example 2: Development of Diagnostic

Volunteer subjects without PD and with PD at different diagnosed stages are tested to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs. Based on the biomarker profile determined, subjects are classified as showing presence or absence of disease and, optionally stage of disease. Profiles are determined using a computerized learning algorithm that, after data analysis, generates a classification algorithm that infers a diagnosis. The inference model is selected to produce a test with a desired sensitivity and specificity.

### III. Example 3: Subject Stratification/Clinical Trial

Volunteer subjects without PD and with PD are tested to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs in neuronally derived extracellular vesicles. Based on the biomarker profile determined, and using a classifier determined in the example above, the subjects are clustered into several test groups. Certain test groups are given a placebo. Other test groups are administered different amounts of a compound in a clinical trial. During and, optionally after administration, the tests are repeated. Collected measurements are analyzed. It is determined that the therapeutic intervention produces a statistically significant change toward normal of biomarker profiles.

### IV. Example 4: Clinical Trial for Drug Candidate that is Neuroprotective for Synucleinopathies

The goal of a Phase II study is to evaluate the safety, tolerability and initial efficacy of pramipexole, given with Aprepitant and with or without and, optionally lovastatin or similarly effective drugs, in patients with PD and related disorders. A sequential treatment, rising-dose, cross-over, out-patient trial in up to 30 patients with PD (PD), Multiple system atrophy (MSA), Lewy body dementia (LBD), or related synucleinopathic disorder is performed. None of the participants is allowed to have been treated with a dopamine agonist or other centrally active pharmaceutical during the 3 months prior to study entry, except for levodopa-carbidopa (Sinemet), which is maintained at a stable dose throughout the trial to a degree considered medically acceptable. Following baseline clinical and laboratory evaluations, including the United PD Rating Scale (UPDRS-Part III) and biomarker determinations comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs, consenting individuals meeting accession criteria are switched from their pre-study PD treatment regimen to one that incudes pramipexole ER and Aprepitant. The pramipexole ER dose is titrated to that which is optimally tolerated (or a maximum of 9 mg/day) and then stably maintained for up to about 12 to 16 weeks. Co-treatment with an additional drug (e.g., a statin) given at its maximum approved dose may then begin for an additional 3 months as deemed clinically appropriate, at which time all subjects are returned to their preadmission treatment regimen. During the trial, baseline efficacy and safety measures were repeated at regular intervals including determination of biomarker levels. Efficacy is determined as a function of statistically significant change toward normal of a biomarker profile.

### V. Example 5: Diagnosis

A subject presents having certain symptoms consistent with PD but, at a preclinical level when still lacking many of the distinguishing clinical features of this illness. Blood is taken from the subject through venipuncture. Amounts of biomarkers including (1) one or more enzymes selected from phosphorylated signaling kinases and/or catalytic enzymes, (2) one or more neurodegeneration-associated proteins in monomeric or oligomeric form, and (3) one or more miRNAs are measured from neuronally derived extracellular vesicles in the blood. A biomarker profile is determined. A diagnostic algorithm classifies the profile to be consistent with a diagnosis of PD. The subject is diagnosed with PD, and is placed on a therapeutic regimen, either a palliative to mitigate symptoms, or treatment directed to the etiology of the disease for purposes of neuroprotection.

### VI. Example 6: Staging

A subject presents with a diagnosis of PD. The medical care provider (e.g., physician) orders a blood test on the subject to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs. Based on the biomarker profile, the medical care professional determines that the subject is at an early stage of PD and thus more responsive to a particular therapeutic intervention.

### VII. Example 7: Prognosis/Progression

A subject presents with a diagnosis of PD. The medical care professional orders first and second blood tests on the subject several months apart to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs. Based on the biomarker profile, the medical care professional determines that the subject's disease is progressing slowly and that the subject is expected to have many years of useful life, even without a risky therapeutic intervention.

### VIII. Example 8: Risk Assessment

A subject presents for a physical exam having no symptoms of a synucleinopathic disease. In this case, this individual is aware of a genetic or environmental risk factor. The medical care professional orders a blood test on the subject to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs. Based on the relatively abnormal biomarker profile of some or all measurable species of biomarkers compared to healthy control individuals, the medical care professional determines that the subject has a low probability of developing PD.

### IX. Example 9: Response to Therapy

A subject presents with a diagnosis of PD. The medical care professional orders initial blood tests on the subject to determine a biomarker profile comprising biomarkers including (1) one or more enzymes selected from phosphorylated signaling kinases and/or catalytic enzymes, (2) one or more neurodegeneration-associated proteins in monomeric or oligomeric form, and (3) one or more miRNAs before treatment commences. After a round of treatment, but before clinical symptoms have changed, the medical care professional orders a second blood test. Based on a change towards normal in the a, the medical care professional determines that the treatment is effective or whether the dose needs to be changed or repeated.

### EXEMPLARY EMBODIMENTS

1. A method for creating a diagnostic index for a neurodegenerative condition, comprising:
a) enriching each biological sample in a collection of biological samples for neuronally derived extracellular vesicles, e.g., microvesicles or exosomes, wherein the collection of biological samples is from subjects in a cohort of subjects, wherein the cohort comprises subjects including:
   (i) a plurality of subjects diagnosed with a neurodegenerative condition at each of one or a plurality of different disease stages, wherein each of the diagnosed subjects has received a putative neuroprotective agent, and/or
   (ii) a plurality of control subjects not diagnosed with the neurodegenerative condition, wherein the biological samples were collected before and again at one or more times during a course of treatment and, optionally, after a course of treatment with the putative neuroprotective agent;
b) isolating protein contents from: whole extracellular vesicles, an internal compartment of the extracellular vesicles, or an extracellular vesicle membrane, from each sample to produce a plurality of biomarker samples;
c) measuring, in each biomarker sample, a set of biomarkers to create a dataset, wherein the set of biomarkers includes:
   (i) a plurality of different signaling kinases; or
   (ii) biomarkers from at least two groups selected from:
      (1) one or more enzymes selected from phosphorylated signaling kinases and/or catalytic enzymes,
      (2) one or more neurodegeneration-associated proteins in monomeric or oligomeric form, and
      (3) one or more miRNAs; and
d) performing an analysis on the dataset to compare differences in the biomarker sets:
   (i) in individual subjects over time to determine a diagnostic algorithm that predicts rates of disease progression or degree of response to the putative neuroprotective agent; or
   (ii) between different subjects to determine a diagnostic algorithm that (1) makes a pathogenic diagnosis, (2) separates clinically similar but etiologically different neurodegenerative disorder subgroups, or (3) predicts whether or the degree to which a subject is likely to respond to the putative neuroprotective agent.

2. The method of any of the preceding embodiments, further comprising, before enriching:
I) providing a cohort of subjects, wherein the cohort comprises subjects including:
(i) a plurality of subjects diagnosed with a neurodegenerative condition at each of a plurality of different disease stages, and/or (ii) a plurality of control subjects;
II) administering to each of the diagnosed subjects a putative neuroprotective agent;
III) before and again at one or more times during and, optionally, after administration of the putative neuroprotective agent, collecting a biological sample from each of the subjects in the cohort.

3. The method of any of the preceding embodiments, further comprising:
e) validating one or more of the diagnostic algorithms against standard clinical measures.

4. The method of any of the preceding embodiments, wherein the biomarkers include (1) one or more phosphorylated signaling kinases and/or catalytic enzymes, one or more (2) one or more neurodegeneration-associated proteins in monomeric or oligomeric form, and (3) one or more miRNAs.

5. The method of any of the preceding embodiments, wherein the enzyme comprises one or more signaling kinases.

6. The method of embodiment 5, wherein at least one of the signaling kinases is a kinase of the PI3K-Akt-mTOR signaling pathway.

7. The method of embodiment 5, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

8. The method of embodiment 5, wherein the at least one signaling kinase is a plurality of the signaling kinases.

9. The method of embodiment 8, wherein a plurality of the signaling kinases are selected from AKT, MAPK3, MEK, mTOR, GSK3B, JNK, MEK 1/2, and PI3K.

10. The method of embodiment 8, wherein the plurality of signaling kinases comprise phosphorylated AKT (e.g., AKT S473 or AKT T308) and phosphorylated MAPK3 (e.g., MAPK T202).

11. The method of embodiment 5, wherein at least one of the signaling kinases is selected from AKT S473; AKT T308; ERK P44; GSK3B S6; GSK3B S9; GSK3 T216; GSK3A S21; MAPK T202; mTOR S2448; mTOR c1/2 T246; mTOR c1/2 S638; JNK 1/2/3; JNK pY183; JNK pY185; MEK 1/2 S217; MEK S221; PI3K p85; PI3K T458; PKB S473; PI3K p55-T199; and PKB T308.

12. The method of embodiment 8, wherein the diagnostic algorithm determines relative amounts of AKT:MAPK.

13. The method of embodiment 1, wherein at least one catalytic enzyme is selected from TH (tyrosine hydroxylase) total, and a phosphorylated form, TH S40, TH S19 and TH S32.

14. The method of any of the preceding embodiments, wherein the diagnostic algorithm is a function of measures of: one or more signaling kinases, one or more catalytic enzymes, one or more neurodegeneration-associated protein forms and one or more microRNAs.

15. The method of any of the preceding embodiments, wherein the diagnostic algorithm is a function of measures of: AKT, phosphorylated tyrosine hydroxylase, an miRNA, MAPK3, and a non-phosphorylated tyrosine hydroxylase.

16. The method of any of the preceding embodiments, wherein the diagnostic algorithm is a function of measures of (AKT, phosphorylated tyrosine hydroxylase, an miRNA) to (MAPK3 and non-phosphorylated tyrosine hydroxylase).

17. The method of any of the preceding embodiments, wherein the diagnostic algorithm is a function of at least a neurodegeneration-associated protein form.

18. The method of embodiment 17, wherein the neurodegeneration-associated protein forms for which the quantitative measures are determined are selected from:
(I) at least one oligomeric form;
(II) a plurality of oligomeric forms;
(III) at least one oligomeric form and at least one monomeric form;
(IV) a plurality of oligomeric forms and at least one monomeric form;
(V) at least one oligomeric form and a plurality of monomeric forms; and
(VI) a plurality of oligomeric forms and a plurality of monomeric forms.

19. The method of any of the preceding embodiments, wherein the diagnostic algorithm is a function of relative measures of (a phosphorylated form of AKT, a phosphorylated form of a second signaling kinase, an oligomeric form of alpha-synuclein, an miRNA) to (a phosphorylated form of MAPK3, a phosphorylated form of a fourth signaling kinase, a non-phosphorylated form of tyrosine hydroxylase).

20. The method of any of the preceding embodiments, wherein the diagnostic algorithm is a function of measures of: AKT, phosphorylated tyrosine hydroxylase, a neurodegeneration-associated protein form, MAPK3, and a non-phosphorylated tyrosine hydroxylase.

21. The method of any of the preceding embodiments, wherein the diagnostic algorithm is a function of relative measures of (AKT, phosphorylated tyrosine hydroxylase, a neurodegeneration-associated protein form) to (MAPK3 and non-phosphorylated tyrosine hydroxylase).

22. The method of any of the preceding embodiments, wherein the diagnostic algorithm is a function of one or more neurodegeneration-associated protein forms and one or more miRNAs.

23. The method of any of the preceding embodiments, wherein the biomarkers comprise (i) an enzyme selected from a signaling kinase and a catalytic enzyme, and (ii) a neurodegeneration-associated protein selected from monomers and oligomers.

24. The method of any of the preceding embodiments, wherein the biomarkers comprise (i) an enzyme selected from a signaling kinase and a catalytic enzyme, and (iii) an miRNA.

25. The method of any of the preceding embodiments, wherein the neurodegeneration-associated protein selected from alpha synuclein, amyloid beta, tau, or huntingtin.

26. The method of any of the preceding embodiments, wherein the oligomeric form of the neurodegeneration-associated protein is a collection of oligomeric forms, e.g., oligomers of alpha synuclein, e.g., alpha synuclein 2-50, e.g., alpha synuclein 4-30, e.g., alpha synuclein 4-20.

27. The method of any of the preceding embodiments, wherein the biomarkers comprise one or more miRNAs selected from 7-5p; 15b-5p; 19b; 22-3p; 24; 27a-3p 24; 29a; 30c-2-3p; 494-3p; 92b-3p; 106b-3p; 122-5p; 124-3p; 122-5p; 132-3p; 138-5p; 142-3p; 146a-5p; 204-5p; 220-3p; 331-5p; 338-3p; 431-5p; 584-5p; 942-5p; and 1468-5p.

28. The method of any of the preceding embodiments, wherein the neurodegenerative condition comprises a synucleinopathic disorder, e.g., Parkinson's disease, or Lewy body dementia.

29. The method of any of the preceding embodiments, wherein the neurodegenerative condition comprises an amyloidopathy, e.g., Alzheimer's Disease, a tauopathy, e.g., Alzheimer's Disease or Huntington's disease.

30. The method of any of the preceding embodiments, wherein protein contents are isolated from an internal compartment of the microsomes.

31. The method of embodiment 3, wherein the standard clinical measures are selected from UPDRS scores, CGI scores and radiologic findings.

32. The method of any of the preceding embodiments, wherein the analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test).

33. The method of any of any of the preceding embodiments, wherein the analysis is executed by computer.

34. The method of embodiment 33, wherein the analysis comprises machine learning.

35. The method of any of the preceding embodiments, wherein the biological sample comprises a venous blood sample.

36. The method of any of the preceding embodiments, wherein the different disease stages comprise one or more of suspected, early, middle, and clinically advanced.

37. The method of any of the preceding embodiments, wherein the times during or after administration are selected from 1, 2, 3 or more months after treatment.

38. The method of any of the preceding embodiments, wherein sample is further enriched for extracellular vesicles from dopamine-producing neurons.

39. The method of any of the preceding embodiments, wherein enriching comprises using one or more brain-specific protein markers.

40. The method of embodiment 39, wherein at least one of the brain-specific markers comprises K1cam.

41. The method of any of the preceding embodiments, wherein isolating comprises washing the extracellular vesicles in each enriched sample to remove surface membrane-bound proteins.

42. The method of embodiment 41, wherein the extracellular vesicles are washed with PBS.

43. The method of any of the preceding embodiments, wherein the forms of the neurodegeneration-associated protein are measured by gel electrophoresis, Western blot or fluorescence techniques.

44. The method of any of the preceding embodiments, wherein the subjects are humans.

45. The method of any of the preceding embodiments, wherein the subjects have been exposed to an environmental condition suspected to be involved in the etiology of (e.g., cause) the neurodegenerative disease (e.g., exposed to paraquat, suspected of causing Parkinson's Disease).

46. The method of any of the preceding embodiments wherein the collection of biological samples comprises at least any of 25, 50, 100, 200, 500 or 1000 samples.

47. A method of developing a diagnostic index that infers the state of the neurodegenerative condition in an individual comprising:
a) providing a dataset comprising, for each of a plurality of subjects, values indicating (1) state of a neurodegenerative condition, and (2) measures of a set of biomarkers, wherein the set of biomarkers includes:
   (i) a plurality of different signaling kinases; or
   (ii) biomarkers from at least two groups selected from:
      (1) one or more enzymes selected from phosphorylated signaling kinases and/or catalytic enzymes,
      (2) one or more neurodegeneration-associated proteins in monomeric or oligomeric form, and
      (3) one or more miRNAs; and
b) performing an analysis on the dataset to develop a model that infers the state of the neurodegenerative condition in an individual.

48. The method of embodiment 47, wherein the analysis is performed by computer.

49. The method of embodiment 47, wherein the analysis is not performed by computer.

50. The method of embodiment 47, wherein the analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test).

51. The method of embodiment 47, wherein the analysis comprises receiving the dataset into computer memory, and training a machine learning algorithm on the dataset with a computer processor.

52. The method of embodiment 51, wherein the machine learning algorithm is selected from: artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting).

53. The method of embodiment 47, wherein the state is selected from diagnosis, stage, prognosis or progression of the neurodegenerative condition.

54. The method of embodiment 47, wherein the state is measured as a categorical variable (e.g., a binary state or one of a plurality of categorical states).

55. The method of embodiment 54, wherein the categories comprise a diagnosis consistent with (e.g., positive or diagnosed as having) having the neurodegenerative condition and inconsistent with (e.g., negative or diagnosed as not having) having the neurodegenerative condition.

56. The method of embodiment 54, wherein the categories comprise different stages of the neurodegenerative condition.

57. The method of embodiment 47, wherein the state is measured as a continuous variable (e.g., on a scale).

58. The method of embodiment 57, wherein the continuous variable is a range is or degrees of the neurodegenerative condition.

59. The method of embodiment 47, wherein the subjects are animals, e.g., fish, avians, amphibians, reptiles, or mammals, e.g., rodents, primates or humans.

60. The method of embodiment 47, wherein the plurality of subjects is at least any of 10, 25, 50, 100, 200, 400 or 800.

61. The method of embodiment 47, wherein, for each subject, the sample for which the quantitative measures are determined are taken at a first time point and the state of the neurodegenerative condition is determined at a second, later time point.

62. The method of embodiment 47, wherein the biological sample comprises blood or a blood fraction (e.g., plasma or serum).

63. The method of embodiment 47, wherein the neurodegenerative condition is a synucleinopathy, e.g., Parkinson's Disease or Lewy Body Dementia.

64. The method of embodiment 47, wherein the neurodegenerative condition is an amyloidopathy, e.g., Alzheimer's Disease, a tauopathy, e.g., Alzheimer's Disease or Huntington's disease.

65. The method of any of embodiments 47-64 preceding embodiments wherein the plurality of subjects is at least any of 25, 50, 100, 200, 500, or 1000 subjects.

66. A method of inferring a risk of developing, a diagnosis of, a stage of, a prognosis of or a progression of a neurodegenerative condition characterized by a neurodegeneration-associated protein, wherein the method comprises:
a) measuring, from a biological sample from a subject that is enriched for neuronally derived extracellular vesicles, e.g., microvesicles or exosomes, a set of biomarkers to create a dataset, wherein the set of biomarkers includes:
   (i) a plurality of different signaling kinases; or
   (ii) biomarkers from at least two groups selected from:
      (1) one or more enzymes selected from phosphorylated signaling kinases and/or catalytic enzymes,
      (2) one or more neurodegeneration-associated proteins in monomeric or oligomeric form, and
      (3) one or more miRNAs; and
b) executing a model, e.g., a model of embodiment 47, on the dataset to infer a risk of developing, a diagnosis of, a stage of, a prognosis of or a progression of the neurodegenerative condition.

67. The method of embodiment 66, wherein at least one of the signaling kinases is a kinase of the PI3K-Akt-mTOR signaling pathway.

68. The method of embodiment 66, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

69. The method of embodiment 66, wherein the neurodegeneration-associated protein selected from alpha synuclein, amyloid beta, tau, or huntingtin.

70. The method of embodiment 66, wherein the oligomeric form of the neurodegeneration-associated protein is a collection of oligomeric forms, e.g., oligomers of alpha synuclein, e.g., alpha synuclein 2-50, e.g., alpha synuclein 4-30, e.g., alpha synuclein 4-20.

71. The method of embodiment 66, wherein at least one of the oligomeric forms comprises a collection of species of the neurodegeneration-associated protein.

72. The method of embodiment 66, wherein the model comprises comparing relative amounts an oligomeric form to monomeric form of the neurodegeneration-associated protein to relative amounts in a statistically significant number of control individuals.

73. The method of embodiment 66, wherein the model comprises detecting a pattern of relative amounts of a plurality of the oligomeric forms from which model the inference is made.

74. The method of embodiment 66, wherein the subject is asymptomatic or preclinical for a neurodegenerative condition.

75. The method of embodiment 66, wherein the subject presents to a healthcare provider, such as a medical care professional, during a routine office visit or as part of a medical care professional's ordinary practice of medicine.

76. The method of embodiment 66, wherein the model is executed by computer.

77. The method of embodiment 66, wherein the model is not executed by computer.

78. A method for determining effectiveness of a therapeutic intervention in treating a neurodegenerative condition, wherein the method comprises:
(a) inferring, in each subject in a population comprising a plurality of subjects, an initial state of a neurodegenerative condition by:
   (1) measuring, from a biological sample from a subject that is enriched for neuronally derived extracellular vesicles, e.g., microvesicles or exosomes, a set of biomarkers to create a dataset, wherein the set of biomarkers includes:
      (i) a plurality of different signaling kinases; or
      (ii) biomarkers from at least two groups selected from:
         (A) one or more enzymes selected from phosphorylated signaling kinases and/or catalytic enzymes,
         (B) one or more neurodegeneration-associated proteins in monomeric or oligomeric form, and
         (C) one or more miRNAs; and
   (2) inferring the initial state using a model, e.g., a model of embodiment 47;
(b) after inferring, administering the therapeutic intervention to the subjects;
(c) after administering, inferring, in each subject individual in the population, a subsequent state of the neurodegenerative condition by:
   (1) measuring, from a biological sample from a subject that is enriched for neuronally derived extracellular vesicles, e.g., microvesicles or exosomes, a set of biomarkers to create a dataset, wherein the set of biomarkers includes:
      (i) a plurality of different signaling kinases; or
      (ii) biomarkers from at least two groups selected from:
         (1) one or more enzymes selected from phosphorylated signaling kinases and/or catalytic enzymes,
         (2) one or more neurodegeneration-associated proteins in monomeric or oligomeric form, and
         (3) one or more miRNAs; and
         (2) inferring the subsequent state using the model; and
(d) based on the initial and subsequent inferences in the population, determining that the therapeutic intervention is effective if the subsequent inferences exhibit a statistically significant change toward a normal state compared with the initial inferences, or that the therapeutic intervention is not effective if the subsequent inferences do not exhibit a statistically significant change compared with the initial inferences toward a normal state.

79. The method of embodiment 78, wherein the therapeutic intervention comprises administration of a drug or combination of drugs.

80. The method of embodiment 78, wherein the population comprises at least 20, at least 50, at least 100, at least 200, at least 500 or at least 1000 subjects, wherein at least 20%, at least 35%, at least 50%, or at least 75% of the subjects initially have elevated amounts of oligomeric forms of the protein relative to amounts of monomeric forms of the protein.

81. The method of embodiment 78, wherein at least 20%, at least 25%, at least 30%, or at least 35%, least 50%, at least 66%, at least 80%, or 100% of the subjects initially have a diagnosis of a neurodegenerative condition.

82. The method of embodiment 78, wherein the inference is made by computer.

83. The method of embodiment 78, wherein the inference is not made by computer.

84. A method for qualifying subjects for a clinical trial of a therapeutic intervention for the treatment or prevention of a neurodegenerative condition comprising:
a) determining that a subject is abnormal with respect with a neurodegenerative condition by:
   (1) measuring, from a biological sample from a subject that is enriched for neuronally derived extracellular vesicles, e.g., microvesicles or exosomes, a set of biomarkers to create a dataset, wherein the set of biomarkers includes;
      (i) a plurality of different signaling kinases; or
      (ii) biomarkers from at least two groups selected from:
         (A) one or more enzymes selected from phosphorylated signaling kinases and/or catalytic enzymes,
         (B) one or more neurodegeneration-associated proteins in monomeric or oligomeric form, and
         (C) one or more miRNAs; and
   (2) executing a model, e.g., a model of embodiment 47, on the profile to infer that the subject is abnormal with respect with the neurodegenerative condition; and
b) enrolling the subject in the clinical trial of a potentially therapeutic intervention for said neurodegenerative condition.

85. The method of embodiment 84, wherein the model is executed by computer.

86. The method of embodiment 84, wherein the model is not executed by computer.

87. A method of monitoring progress of a subject on a therapeutic intervention for a neurodegenerative condition comprising:
(a) inferring, in the subject, an initial state of a neurodegenerative condition by:
   (1) determining, from a biological sample from a subject that is enriched for neuronally derived extracellular vesicles, e.g., microvesicles or exosomes, measures of a set of biomarkers, wherein the set of biomarkers includes:
      (i) a plurality of different signaling kinases; or
      (ii) biomarkers from at least two groups selected from:
         (A) one or more enzymes selected from phosphorylated signaling kinases and/or catalytic enzymes,
         (B) one or more neurodegeneration-associated proteins in monomeric or oligomeric form, and
         (C) one or more miRNAs; and
   (2) executing a model, e.g., a model of embodiment 47, to infer an initial state of the neurodegenerative condition;
(b) after inferring, administering the therapeutic intervention to the subject;
(c) after administering, inferring, in the subject, a subsequent state of the neurodegenerative condition by:
   (1) determining, from a biological sample from a subject that is enriched for neuronally derived microsomal particles, a biomarker profile comprising amounts of each of a plurality of different signaling kinases to create a dataset; and
   (2) executing a model, e.g. a model of embodiment 47, to infer a subsequent state of the neurodegenerative condition; and
(d) based on the initial and subsequent state inferences, determining that the subject is responding positively to the therapeutic intervention if the subsequent inference exhibits a change toward a normal state compared with the initial inferences, or that the therapeutic intervention is not effective if the subsequent inferences do not exhibit a change compared with the initial inferences toward a normal state.

88. The method of embodiment 87, wherein the model is executed by computer.

89. The method of embodiment 87, wherein the model is not executed by computer.

90. A method comprising:
(a) determining, by the method of embodiment 66, that a subject has a neurodegenerative condition, and
(b) administering to the subject a palliative or neuroprotective therapeutic intervention efficacious to treat the condition.

91. The method of embodiment 90, wherein the therapeutic intervention moves a biomarker profile of the subject toward normal, wherein a movement toward normal indicates neuroprotection.

92. A method comprising administering to a subject determined by the method of embodiment 66 to have an abnormal pattern of biomarkers, a palliative or neuroprotective therapeutic intervention effective to treat the condition.

93. The method of embodiment 92, wherein the subject is asymptomatic or preclinical for the neurodegenerative condition.

94. A kit comprising reagents sufficient to detect either:
(1) at least one of signaling kinase and at least one oligomeric form of a neurodegeneration-associated protein; or
(2) a plurality of different signaling kinases.

95. The kit of embodiment 94, wherein the reagents comprise antibodies.

96. A method of inferring a risk of developing, a diagnosis of, a stage of, a prognosis of or a progression of a neurodegenerative condition, wherein the method comprises:
a) measuring, from a biological sample from a subject that is enriched for neuronally derived extracellular vesicles, e.g., microvesicles or exosomes, a set of biomarkers to create a dataset, wherein the set of biomarkers includes:
   (i) a plurality of different signaling kinases; or
   (ii) biomarkers from at least two groups selected from:
      (A) one or more enzymes selected from phosphorylated signaling kinases and/or catalytic enzymes,
      (B) one or more neurodegeneration-associated proteins in monomeric or oligomeric form, and
      (C) one or more miRNAs; and
b) correlating the dataset with a risk of developing, a diagnosis of, a stage of, a prognosis of or a progression of the neurodegenerative condition.

97. A method comprising:
(a) identifying a subject having a neurodegenerative condition or likely to positively respond to a treatment for a neurodegenerative condition, wherein identifying comprises:
   (1) measuring, in a sample from the subject enriched for neuronally derived extracellular vesicles (e.g., from the internal contents of the extracellular vesicles), a set of biomarkers, to create a biomarker profile, wherein the set of biomarkers includes:
      (i) a plurality of different signaling kinases; or
      (ii) biomarkers from at least two groups selected from:
         (A) one or more enzymes selected from phosphorylated signaling kinases and/or catalytic enzymes,
         (B) one or more neurodegeneration-associated proteins in monomeric or oligomeric form, and
         (C) one or more miRNAs; and
   (2) determining, based on an abnormal biomarker profile, that the subject suffers from the neurodegenerative condition; and
(b) administering to the identified subject, an effective amount of a pharmaceutical composition to treat the neurodegenerative condition.

98. The method of embodiment 97, wherein the neurodegenerative condition is a synucleopathic condition, and the pharmaceutical composition comprises comprising a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)).

99. The method of embodiment 97, wherein the synucleopathic condition is Parkinson's Disease.

100. The method of embodiment 99, wherein the pharmaceutical composition comprises a dopamine agonist.

101. The method of embodiment 100, wherein the pharmaceutical composition further comprises an NK1-antagogonist.

102. The method of embodiment 101, wherein the dopamine agonist is 6-propylamino-4,5,6,7-tetrahydro-1,3-benzothiazole-2-amine and the NK1-antagonist is aprepitant or rolapitant.

103. The method of embodiment 100, wherein the pharmaceutical composition further comprises a 5HT3-antagonist.

104. The method of embodiment 103, wherein the dopamine agonist is 6-propylamino-4,5,6,7-tetrahydro-1,3-benzothiazole-2-amine and the 5HT3 antagonist is ondansetron hydrochloride dihydrate.

105. A method comprising administering to a subject characterized as having a biomarker profile indicative of a neurodegenerative condition or being likely to positively respond to a treatment for a neurodegenerative condition, an effective amount of a pharmaceutical composition to treat the neurodegenerative condition; wherein the biomarker panel comprises set of biomarkers includes one or a plurality of signaling kinases and, optionally, at least one oligomeric form of a neurodegeneration-associated protein measured from a sample from the subject enriched for neuronally derived extracellular vesicles (e.g., from the internal contents of the extracellular vesicles).

106. The method of embodiment 105, wherein the neurodegenerative condition is Parkinson's Disease, and wherein the pharmaceutical composition comprises a dopamine agonist.

107. A kit comprising reagents sufficient to detect either:
(1) at least one of signaling kinase,
(2) at least one catalytic enzyme,
(3) at least one oligomeric form of a neurodegeneration-associated protein, and
(4) at least one miRNA.

108. A method comprising:
at a computer system comprising at least one processor and a memory storing at least one program for execution by the at least one processor:
a) obtaining biomarker data in electronic form for a plurality of biomarkers from a biological sample from each of at least 25, 50, 100, 200, 500 or 1000 subjects, wherein:
   (i) the subjects comprise (i) a plurality of subjects diagnosed with a neurodegenerative condition at each of one or a plurality of different disease stages, wherein each of the diagnosed subjects has received a putative neuroprotective agent, and (ii) a plurality of control subjects not diagnosed with the neurodegenerative condition;
   (ii) the samples are enriched for neuronally derived exosomes; and
   (iii) the biomarker data comprises measures of:
   (i) a plurality of different signaling kinases; or
   (ii) biomarkers from at least two groups selected from:
   (1) one or more enzymes selected from phosphorylated signaling kinases and/or catalytic enzymes,
   (2) one or more neurodegeneration-associated proteins in monomeric or oligomeric form, and
   (3) one or more miRNAs;
b) executing a learning algorithm, using computer logic, to produce a model that:
   (i) in individual subjects over time predicts rates of disease progression or degree of response to the putative neuroprotective agent; or
   (ii) between different subjects (1) makes a pathogenic diagnosis, (2) separates clinically similar but etiologically different neurodegenerative disorder subgroups, or (3) predicts whether or the degree to which a subject is likely to respond to the putative neuroprotective agent.

109. A method comprising:
at a computer system comprising at least one processor and a memory storing at least one program for execution by the at least one processor:
a) obtaining biomarker data in electronic form for a plurality of biomarkers from a biological sample from a subject, wherein:
   (i) the samples are enriched for neuronally derived exosomes; and
   (ii) the biomarker data comprises measures of:
   (i) a plurality of different signaling kinases; or
   (ii) biomarkers from at least two groups selected from:
      (1) one or more enzymes selected from phosphorylated signaling kinases and/or catalytic enzymes,
      (2) one or more neurodegeneration-associated proteins in monomeric or oligomeric form, and
      (3) one or more miRNAs;
b) executing a model, using computer logic, that:
   (i) in individual subjects over time predicts rates of disease progression or degree of response to the putative neuroprotective agent; or
   (ii) between different subjects (1) makes a pathogenic diagnosis, (2) separates clinically similar but etiologically different neurodegenerative disorder subgroups, or (3) predicts whether or the degree to which a subject is likely to respond to the putative neuroprotective agent; and
c) outputting the prediction to an electronic device accessible by the subject.

As used herein, the following meanings apply unless otherwise specified. The words “can” and “may” are used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality”, and, therefore, contemplates the use of the term “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims.

All publications, patents, and patent applications mentioned in this specification are herein incorporated by reference to the same extent as if each individual publication, patent, or patent application was specifically and individually indicated to be incorporated by reference.

1. A method for creating a diagnostic index for a neurodegenerative condition, comprising:
a) enriching each biological sample in a collection of biological samples for neuronally derived extracellular vesicles, e.g., microvesicles or exosomes, wherein the collection of biological samples is from subjects in a cohort of subjects, wherein the cohort comprises subjects including:
(i) a plurality of subjects diagnosed with a neurodegenerative condition at each of one or a plurality of different disease stages, wherein each of the diagnosed subjects has received a putative neuroprotective agent, and/or
(ii) a plurality of control subjects not diagnosed with the neurodegenerative condition,
wherein the biological samples were collected before and again at one or more times during a course of treatment and, optionally, after a course of treatment with the putative neuroprotective agent;
b) isolating protein contents from: whole extracellular vesicles, an internal compartment of the extracellular vesicles, or an extracellular vesicle membrane, from each sample to produce a plurality of biomarker samples;
c) measuring, in each biomarker sample, a set of biomarkers to create a dataset, wherein the set of biomarkers includes:
(i) a plurality of different signaling kinases; or
(ii) biomarkers from at least two groups selected from:
(1) one or more enzymes selected from phosphorylated signaling kinases and/or catalytic enzymes,
(2) one or more neurodegeneration-associated proteins in monomeric or oligomeric form, and
(3) one or more miRNAs; and
d) performing an analysis on the dataset to compare differences in the biomarker sets:
(i) in individual subjects over time to determine a diagnostic algorithm that predicts rates of disease progression or degree of response to the putative neuroprotective agent; or
(ii) between different subjects to determine a diagnostic algorithm that (1) makes a pathogenic diagnosis, (2) separates clinically similar but etiologically different neurodegenerative disorder subgroups, or (3) predicts whether or the degree to which a subject is likely to respond to the putative neuroprotective agent.

2.-46. (canceled)

47. A method of developing a diagnostic index that infers the state of the neurodegenerative condition in an individual comprising:
a) providing a dataset comprising, for each of a plurality of subjects, values indicating (1) state of a neurodegenerative condition, and (2) measures of a set of biomarkers, wherein the set of biomarkers includes:
(i) a plurality of different signaling kinases; or
(ii) biomarkers from at least two groups selected from:
(1) one or more enzymes selected from phosphorylated signaling kinases and/or catalytic enzymes,
(2) one or more neurodegeneration-associated proteins in monomeric or oligomeric form, and
(3) one or more miRNAs; and
b) performing an analysis on the dataset to develop a model that infers the state of the neurodegenerative condition in an individual.

48.-65. (canceled)

66. A method of inferring a risk of developing, a diagnosis of, a stage of, a prognosis of or a progression of a neurodegenerative condition characterized by a neurodegeneration-associated protein, wherein the method comprises:
a) measuring, from a biological sample from a subject that is enriched for neuronally derived extracellular vesicles, e.g., microvesicles or exosomes, a set of biomarkers to create a dataset, wherein the set of biomarkers includes:
(i) a plurality of different signaling kinases; or
(ii) biomarkers from at least two groups selected from:
(1) one or more enzymes selected from phosphorylated signaling kinases and/or catalytic enzymes,
(2) one or more neurodegeneration-associated proteins in monomeric or oligomeric form, and
(3) one or more miRNAs; and
b) executing a model, e.g., a model of claim 47, on the dataset to infer a risk of developing, a diagnosis of, a stage of, a prognosis of or a progression of the neurodegenerative condition.

67. The method of claim 66, wherein at least one of the signaling kinases is a kinase of the PI3K-Akt-mTOR signaling pathway.

68. The method of claim 66, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

69. The method of claim 66, wherein the neurodegeneration-associated protein selected from alpha synuclein, amyloid beta, tau, or huntingtin.

70. The method of claim 66, wherein the oligomeric form of the neurodegeneration-associated protein is a collection of oligomeric forms, e.g., oligomers of alpha synuclein, e.g., alpha synuclein 2-50, e.g., alpha synuclein 4-30, e.g., alpha synuclein 4-20.

71. The method of claim 66, wherein at least one of the oligomeric forms comprises a collection of species of the neurodegeneration-associated protein.

72. The method of claim 66, wherein the model comprises comparing relative amounts an oligomeric form to monomeric form of the neurodegeneration-associated protein to relative amounts in a statistically significant number of control individuals.

73. The method of claim 66, wherein the model comprises detecting a pattern of relative amounts of a plurality of the oligomeric forms from which model the inference is made.

74. The method of claim 66, wherein the subject is asymptomatic or preclinical for a neurodegenerative condition.

75.-77. (canceled)

78. A method for determining effectiveness of a therapeutic intervention in treating a neurodegenerative condition, wherein the method comprises:
(a) inferring, in each subject in a population comprising a plurality of subjects, an initial state of a neurodegenerative condition by:
(1) measuring, from a biological sample from a subject that is enriched for neuronally derived extracellular vesicles, e.g., microvesicles or exosomes, a set of biomarkers to create a dataset, wherein the set of biomarkers includes:
(i) a plurality of different signaling kinases; or
(ii) biomarkers from at least two groups selected from:
(A) one or more enzymes selected from phosphorylated signaling kinases and/or catalytic enzymes,
(B) one or more neurodegeneration-associated proteins in monomeric or oligomeric form, and
(C) one or more miRNAs; and
(2) inferring the initial state using a model, e.g., a model of claim 47;
(b) after inferring, administering the therapeutic intervention to the subjects;
(c) after administering, inferring, in each subject individual in the population, a subsequent state of the neurodegenerative condition by:
(1) measuring, from a biological sample from a subject that is enriched for neuronally derived extracellular vesicles, e.g., microvesicles or exosomes, a set of biomarkers to create a dataset, wherein the set of biomarkers includes:
(i) a plurality of different signaling kinases; or
(ii) biomarkers from at least two groups selected from:
(1) one or more enzymes selected from phosphorylated signaling kinases and/or catalytic enzymes,
(2) one or more neurodegeneration-associated proteins in monomeric or oligomeric form, and
(3) one or more miRNAs; and
(4) inferring the subsequent state using the model; and
(d) based on the initial and subsequent inferences in the population, determining that the therapeutic intervention is effective if the subsequent inferences exhibit a statistically significant change toward a normal state compared with the initial inferences, or that the therapeutic intervention is not effective if the subsequent inferences do not exhibit a statistically significant change compared with the initial inferences toward a normal state.

79. The method of claim 78, wherein the therapeutic intervention comprises administration of a drug or combination of drugs.

80. The method of claim 78, wherein the population comprises at least 20, at least 50, at least 100, at least 200, at least 500 or at least 1000 subjects, wherein at least 20%, at least 35%, at least 50%, or at least 75% of the subjects initially have elevated amounts of oligomeric forms of the protein relative to amounts of monomeric forms of the protein.

81. The method of claim 78, wherein at least 20%, at least 25%, at least 30%, or at least 35%, least 50%, at least 66%, at least 80%, or 100% of the subjects initially have a diagnosis of a neurodegenerative condition.

82.-86. (canceled)

87. A method of monitoring progress of a subject on a therapeutic intervention for a neurodegenerative condition comprising:
(a) inferring, in the subject, an initial state of a neurodegenerative condition by:
(1) determining, from a biological sample from a subject that is enriched for neuronally derived extracellular vesicles, e.g., microvesicles or exosomes, measures of a set of biomarkers, wherein the set of biomarkers includes:
(i) a plurality of different signaling kinases; or
(ii) biomarkers from at least two groups selected from:
(A) one or more enzymes selected from phosphorylated signaling kinases and/or catalytic enzymes,
(B) one or more neurodegeneration-associated proteins in monomeric or oligomeric form, and
(C) one or more miRNAs; and
(2) executing a model, e.g., a model of claim 47, to infer an initial state of the neurodegenerative condition;
(b) after inferring, administering the therapeutic intervention to the subject;
(c) after administering, inferring, in the subject, a subsequent state of the neurodegenerative condition by:
(1) determining, from a biological sample from a subject that is enriched for neuronally derived microsomal particles, a biomarker profile comprising amounts of each of a plurality of different signaling kinases to create a dataset; and
(2) executing a model, e.g. a model of claim 47, to infer a subsequent state of the neurodegenerative condition; and
(d) based on the initial and subsequent state inferences, determining that the subject is responding positively to the therapeutic intervention if the subsequent inference exhibits a change toward a normal state compared with the initial inferences, or that the therapeutic intervention is not effective if the subsequent inferences do not exhibit a change compared with the initial inferences toward a normal state.

88.-95. (canceled)

96. A method of inferring a risk of developing, a diagnosis of, a stage of, a prognosis of or a progression of a neurodegenerative condition, wherein the method comprises:
a) measuring, from a biological sample from a subject that is enriched for neuronally derived extracellular vesicles, e.g., microvesicles or exosomes, a set of biomarkers to create a dataset, wherein the set of biomarkers includes:
(i) a plurality of different signaling kinases; or
(ii) biomarkers from at least two groups selected from:
(A) one or more enzymes selected from phosphorylated signaling kinases and/or catalytic enzymes,
(B) one or more neurodegeneration-associated proteins in monomeric or oligomeric form, and
(C) one or more miRNAs; and
b) correlating the dataset with a risk of developing, a diagnosis of, a stage of, a prognosis of or a progression of the neurodegenerative condition.

97. A method comprising:
(a) identifying a subject having a neurodegenerative condition or likely to positively respond to a treatment for a neurodegenerative condition, wherein identifying comprises:
(1) measuring, in a sample from the subject enriched for neuronally derived extracellular vesicles (e.g., from the internal contents of the extracellular vesicles), a set of biomarkers, to create a biomarker profile, wherein the set of biomarkers includes:
(i) a plurality of different signaling kinases; or
(ii) biomarkers from at least two groups selected from:
(A) one or more enzymes selected from phosphorylated signaling kinases and/or catalytic enzymes,
(B) one or more neurodegeneration-associated proteins in monomeric or oligomeric form, and
(C) one or more miRNAs; and
(2) determining, based on an abnormal biomarker profile, that the subject suffers from the neurodegenerative condition; and
(b) administering to the identified subject, an effective amount of a pharmaceutical composition to treat the neurodegenerative condition.

98. The method of claim 97, wherein the neurodegenerative condition is a synucleopathic condition, and the pharmaceutical composition comprises comprising a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)).

99. The method of claim 97, wherein the synucleopathic condition is Parkinson's Disease.

100. The method of claim 99, wherein the pharmaceutical composition comprises a dopamine agonist.

101. The method of claim 100, wherein the pharmaceutical composition further comprises an NK1-antagogonist.

102. The method of claim 101, wherein the dopamine agonist is 6-propylamino-4,5,6,7-tetrahydro-1,3-benzothiazole-2-amine and the NK1-antagonist is aprepitant or rolapitant.

103. The method of claim 100, wherein the pharmaceutical composition further comprises a 5HT3-antagonist.

104. The method of claim 103, wherein the dopamine agonist is 6-propylamino-4,5,6,7-tetrahydro-1,3-benzothiazole-2-amine and the 5HT3 antagonist is ondansetron hydrochloride dihydrate.

105.-109. (canceled)


SEQUENCE LISTING (1 sequence)

SEQ ID NO: 1 - protein, 42 aa, Unknown [sequence omitted]